SEQUENCE LISTING (2 sequences)

SEQ ID NO: 1 - protein, 107 aa, Homo sapiens [sequence omitted]

SEQ ID NO: 2 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2020/0190138 A1
(43) Pub. Date: Jun. 18, 2020

(54) PROTEIN PURIFICATION WITH PROTEIN L

(71) Applicant: CHUGAI SEIYAKU KABUSHIKI KAISHA, Tokyo (JP)

(72) Inventors: Chen CHEN, Synapse (SG); Yuichiro SHIMIZU, Synapse (SG); Tetsuya WAKABAYASHI, Shizuoka (JP)

(21) Appl. No.: 16/488,746

(22) PCT Filed: Feb. 27, 2018
(86) PCT No.: PCT/JP2018/007280

§ 371 Date: Aug. 26, 2019

(30) Foreign Application Priority Data
Feb. 28, 2017 (JP) 2017-036614
Dec. 25, 2017 (JP) 2017-247614

(51) Int. Cl. C07K 1/22

(52) U.S. Cl. CPC C07K 1/22; C07K 16/32

(57) ABSTRACT

The invention provides methods of purifying and/or producing a protein. In some embodiments, a method of the present invention comprises the step of eluting a protein from a Protein L matrix by lowering a conductivity. In some embodiments, the protein is an antibody. The invention also provides an antibody. In some embodiments, an antibody of the present invention comprises a light chain, which comprises a kappa variable region and a lambda constant

### CROSS-REFERENCE TO RELATED APPLICATIONS

This application is a U.S. National Phase of PCT Application No. PCT/JP2018/007280, filed Feb. 27, 2018, which claims the benefit of Japanese Patent Application No. 2017-036614, filed Feb. 28, 2017, and Japanese Patent Application No. 2017-247614, filed Dec. 25, 2017, each of which is incorporated herein by reference in its entirety.

### REFERENCE TO SEQUENCE LISTING SUBMITTED ELECTRONICALLY

The content of the electronically submitted sequence listing (Name: 6663.0118) Sequence_Listing.txt; Size: 2.20 kilobytes; and Date of Creation: Aug. 26, 2019) filed with the application is incorporated herein by reference in its entirety.

### TECHNICAL FIELD

The present invention relates to methods of purifying proteins with Protein L.

### BACKGROUND ART

There are some previous reports on producing bispecific antibodies. In general, a bispecific antibody is composed of two types of heavy chains and two types of light chains. When trying to recombinantly produce a bispecific antibody by expressing those four components together, it usually leads to a difficulty that ten types of different antibodies can be produced due to the mismatched combinations of the two heavy and two light chains. In that case, it becomes necessary to isolate a single bispecific antibody of interest from a mixture of the ten types of antibodies. To improve the efficiency of producing a bispecific antibody, several methods to promote heterodimerization of two heavy chains have been reported so far, which include, for example, introduction of amino acid substitutions into the heavy chains (see, e.g., PTL 1, PTL 2, and PTL 3). Meanwhile, there is also another need to develop a method to efficiently remove antibodies with VH and VL pairs mismatched.

Protein L was first isolated from bacterial species Peptostreptococcus magnus and was found to bind to immunoglobulins (see, e.g., NPL 1). The discovery of Protein L complemented the other widely used immunoglobulin (Ig)-binding reagents, Protein A and Protein G, for purification, detection and immobilisation of antibodies. Protein L has been reported to bind to kappa light chains of immunoglobulins such as IgG, IgM, IgE, IgD, and IgA derived from mammalian species such as human, rabbit, porcine, mouse, and rat. Studies have shown that the major binding sites of Protein L are comprised within the variable regions of the kappa light chains (see, e.g., NPL 2). More specifically, Protein L has been shown to bind with high affinity to certain subgroups of kappa light chains. For example, it binds to human V kappa I, V kappa III and V kappa IV subgroups but does not bind to the V kappa II subgroup. Binding of mouse immunoglobulins is restricted to those having V kappa I light chains. This unique location of its binding site allows Protein L to bind to antibody fragments as well, such as Fab, Fab′, F(ab′)₂, Fv, and scFv, only if they have a variable region of the certain types of kappa light chains. The crystal structure of Protein L in complex with Fab has also been solved (see, e.g., NPL 3).

It is said that about 75% of the antibodies produced by healthy humans have a kappa light chain. In addition, many therapeutic monoclonal antibodies and antibody fragments contain kappa light chains. In recent years, several approaches have also been attempted to purify bispecific antibodies comprising a kappa light chain using Protein L in combination with certain antibody modification technologies (see, e.g., PTL 4 and PTL 5).
[PTL 1] WO1996/027011
[PTL 2] WO2006/106905
[PTL 3] WO2009/089004
[PTL 4] WO2013/088259
[PTL 5] WO2017/005649

### Non-Patent Literature

[NPL 1] Bjorck L, (1988) J Immunol, 140(4): 1194-1197
[NPL 2] Nilson et al, (1992) J Biol Chem, 267(4): 2234-2239
[NPL 3] Graille et al, (2001) Structure, 9(8): 679-687

### SUMMARY OF INVENTION

### Technical Problem

An objective of the present invention is to provide methods of purifying a protein.

### Solution to Problem

The invention provides methods of purifying a protein.

In some embodiments, a method of the present invention comprises the step of eluting at least two different proteins from a Protein L matrix by lowering a conductivity, wherein each of the proteins comprises a different number of Protein L binding motifs.

In some embodiments, a method of the present invention comprises the steps of:
(a) contacting a solution comprising at least two different proteins with a Protein L matrix at a certain conductivity so that the proteins are bound to the Protein L matrix, and
(b) eluting the bound proteins from the Protein L matrix by lowering the conductivity, wherein each of the proteins comprises a different number of Protein L binding motifs.

In some embodiments, a protein comprising a certain number of the Protein L binding motifs is separated from proteins comprising a different number of the Protein L binding motifs. In some embodiments, a protein comprising one Protein L binding motif is separated from proteins comprising two or more Protein L binding motifs.

In some embodiments, the Protein L binding motif is an antibody kappa chain variable region or a fragment thereof which has a binding ability to Protein L. In further embodiments, the antibody kappa chain variable region is selected from the group consisting of human variable kappa subgroup 1 (VK1), human variable kappa subgroup 3 (VK3), human variable kappa subgroup 4 (VK4), mouse variable kappa subgroup 1 (VK1), and variants thereof.

In some embodiments, any one of the proteins is a monomeric protein comprising a single polypeptide or a multimeric protein comprising two or more polypeptides. In some embodiments, any one of the proteins is an antibody. In certain embodiments, the antibody is a whole antibody or an antibody fragment. In certain embodiments, the antibody is a monospecific antibody or a multispecific antibody.

In some embodiments, the antibody comprises two light chains, one of which comprises a Protein L binding motif. In further embodiments, the antibody comprises two light chains, the other of which comprises a Protein L non-binding motif. In some embodiments, two heavy chains of the antibody are identical or non-identical.

In some embodiments, the solution comprises:
(i) an antibody comprising two light chains, one of which comprises a Protein L binding motif, and the other of which comprises a Protein L non-binding motif, and
(ii) an antibody comprising two light chains, both of which comprise a Protein L binding motif.

In some embodiments, the solution comprises:
(i) an antibody comprising two light chains, one of which comprises a Protein L binding motif, and the other of which comprises a Protein L non-binding motif,
(ii) an antibody comprising two light chains, both of which comprise a Protein L binding motif, and
(iii) an antibody comprising two light chains, both of which comprise a Protein L non-binding motif.

In some embodiments, at least one of the proteins is eluted from the Protein L matrix at a conductivity between 0.01 and 16 mS/cm. In further embodiments, a protein comprising one Protein L binding motif is eluted from the Protein L matrix at a conductivity between 0.01 and 16 mS/cm. In some embodiments, the conductivity is reduced in a gradient manner or in a stepwise manner during the elution step.

In some embodiments, at least one of the proteins is eluted from the Protein L matrix at an acidic pH. In further embodiments, at least one of the proteins is eluted from the Protein L matrix at a pH between 2.4 and 3.3. In further embodiments, a protein comprising one Protein L binding motif is eluted from the Protein L matrix at a pH between 2.4 and 3.3. In some embodiments, the pH remains constant or substantially unchanged during the elution step.

The invention also provides methods of producing a protein.

In some embodiments, a method of the present invention comprises the steps of:
(a) eluting at least two different proteins from a Protein L matrix by lowering a conductivity, and
(b) collecting one of the eluted proteins,
   wherein each of the proteins comprises a different number of Protein L binding motifs.

In some embodiments, a method of the present invention comprises the steps of:
(a) contacting a solution comprising at least two different proteins with a Protein L matrix at a certain conductivity so that the proteins are bound to the Protein L matrix,
(b) eluting the bound proteins from a Protein L matrix by lowering the conductivity, and
(c) collecting one of the eluted proteins,
   wherein each of the proteins comprises a different number of Protein L binding motifs.

In some embodiments, a method of the present invention comprises the steps of:
(a) culturing cells under conditions suitable for expression of a polypeptide comprising at least one Protein L binding motif,
(b) collecting a solution comprising at least two different proteins expressed in the cells, wherein each of the proteins comprises a different number of the polypeptide,
(c) contacting the solution with a Protein L matrix at a certain conductivity so that the proteins are bound to the Protein L matrix,
(d) eluting the bound proteins from the Protein L matrix by lowering the conductivity
(e) collecting one of the eluted proteins.

In some embodiments, a method of the present invention comprises the steps of:
(a) isolating a nucleic acid which encodes a polypeptide comprising at least one Protein L binding motif,
(b) transforming host cells with an expression vector comprising the nucleic acid,
(c) culturing the host cells under conditions suitable for expression of the polypeptide,
(d) collecting a solution comprising at least two different proteins expressed in the host cells, wherein each of the proteins comprises a different number of the polypeptides,
(e) contacting the solution with a Protein L matrix at a certain conductivity so that the proteins are bound to the Protein L matrix, and
(f) eluting the bound proteins from the Protein L matrix by lowering the conductivity, and
(g) collecting one of the eluted proteins.

The invention also provides an antibody.

In some embodiments, the antibody comprises a light chain, which comprises a kappa variable region and a lambda constant region. In further embodiments, the antibody comprises another light chain, which comprises any one of (i) a kappa variable region and a kappa constant region, (ii) a lambda variable region and a lambda constant region, (iii) a kappa variable region and a lambda constant region, or (iv) a lambda variable region and a kappa constant region. In certain embodiments, the antibody is a multispecific antibody.

The present invention provides:
[1] A method of purifying a protein comprising the step of eluting at least two different proteins from a Protein L matrix by lowering a conductivity, wherein each of the proteins comprises a different number of Protein L binding motifs.
[2] The method of [1], wherein one of the proteins which comprises a certain number of Protein L binding motifs is separated from the other protein(s) in the elution step.
[3] The method of [1] or [2], wherein the Protein L binding motif is an antibody kappa chain variable region or a fragment thereof which has a binding ability to Protein L.
[4] The method of [3], wherein the antibody kappa chain variable region is selected from the group consisting of human variable kappa subgroup 1 (VK1), human variable kappa subgroup 3 (VK3), human variable kappa subgroup 4 (VK4), mouse variable kappa subgroup 1 (VK1), and variants thereof.
[5] The method of any one of [1] to [4], wherein any one of the proteins is an antibody.
[6] The method of [5], wherein the antibody is a whole antibody or an antibody fragment.
[7] The method of [5], wherein the antibody is a monospecific antibody or a multispecific antibody.
[8] The method of [5], wherein the at least two different proteins comprise:
   (i) an antibody comprising two light chains, one of which comprises a Protein L binding motif, and the other of which comprises a Protein L non-binding motif, and
   (ii) an antibody comprising two light chains, both of which comprise a Protein L binding motif.
[9] The method of any one of [1] to [8], wherein at least one of the proteins is eluted from the Protein L matrix at a conductivity between 0.01 and 16 mS/cm.
[10] The method of any one of [1] to [9], wherein the conductivity is reduced in a gradient manner or in a stepwise manner during the elution step.
[11] The method of any one of [1] to [10], wherein at least one of the proteins is eluted from the Protein L matrix at an acidic pH.
[12] The method of [11], wherein at least one of the proteins is eluted from the Protein L matrix at a pH between 2.4 and 3.3.
[13] The method of [11] or [12], wherein the pH remains constant or substantially unchanged during the elution step.
[14] A method of producing a protein comprising the steps of:
   (a) eluting at least two different proteins from a Protein L matrix by lowering a conductivity, and
   (b) collecting one of the eluted proteins, wherein each of the proteins comprises a different number of Protein L binding motifs.
[15] An antibody comprising a light chain, which comprises a kappa variable region and a lambda constant region.

### BRIEF DESCRIPTION OF DRAWINGS

FIG. 1 illustrates schematic representation of the structures of different antibodies used in the experiments. Ab #1, Ab #3, Ab #5, and Ab #7 are bispecific antibodies composed of two different heavy chain polypeptides and two different light chain polypeptides. Ab #2, Ab #4, Ab #6, and Ab #8 are monospecific antibodies composed of two copies of unique heavy chain and light chain polypeptides. Ab #3 Ab #4, Ab #7, and Ab #8 have kappa variable domains fused to a kappa constant domain and/or lambda variable domains fused to lambda constant domain. Ab #1 and Ab #5 have one arm composed of kappa variable domain fused to lambda constant domain. Ab #2 and Ab #6 have both arms composed of kappa variable domain fused to lambda constant domain. Ab #9 is a one-arm antibody derived from Ab #8. Ab #10 is a bispecific antibody consisting of two single chain variable fragments with one kappa variable domain and one lambda variable domain.

FIGS. 2A-2D illustrate identification of antibodies by using CIEX method, as described in Example 4. FIG. 2A is a graph depicting an overlay of the representative UV-trace profiles of Ab #1 and Ab #2. FIG. 2B is a graph depicting an overlay of the representative UV-trace profiles of Ab #3 and Ab #4. FIG. 2C is a graph depicting an overlay of the representative UV-trace profiles of Ab #5 and Ab #6. FIG. 2D is a graph depicting an overlay of the representative UV-trace profiles of Ab #7 and Ab #8.

FIGS. 3A-3D illustrate separation of Ab #1 and Ab #2 by conductivity gradient in pH 2.4, 2.7, 3.0, and 3.3, as described in Example 5. FIG. 3A is a graph depicting a representative UV-trace profile of Protein L affinity chromatography using salt gradient elution from 100 mM NaCl to 0 mM at pH 2.4. FIG. 3B is a graph depicting a representative UV-trace profile of Protein L affinity chromatography using salt gradient elution from 100 mM NaCl to 0 mM at pH 2.7. FIG. 3C is a graph depicting a representative UV-trace profile of Protein L affinity chromatography using salt gradient elution from 100 mM NaCl to 0 mM at pH 3.0.

FIG. 3D is a graph depicting a representative UV-trace profile of Protein L affinity chromatography using salt gradient elution from 100 mM NaCl to 0 mM at pH 3.3.

FIGS. 4A-4B illustrate separation of Ab #1 and Ab #2 by two-step purification in pH 2.7 and 3.0, as described in Example 6. FIG. 4A is a graph depicting a representative UV-trace profile of Protein L affinity chromatography using salt step elution at pH 2.7. A table summarizing the content of each peak is present. FIG. 4B is a graph depicting a representative UV-trace profile of Protein L affinity chromatography using salt step elution at pH 3.0. A table summarizing the content of each peak is present.

FIGS. 5A-5B illustrate separation of Ab #3 and Ab #4 by conductivity gradient and step in pH 2.7, as described in Example 7. FIG. 5A is a graph depicting a representative UV-trace profile of Protein L affinity chromatography using salt gradient elution from 100 mM NaCl to 0 mM at pH 2.7. FIG. 5B is a graph depicting a representative UV-trace profile of Protein L affinity chromatography using salt step elution at pH 2.7. A table summarizing the content of each peak is present.

FIGS. 6A-6D illustrate separation of Ab #5 and Ab #6 by conductivity gradient in pH 2.4, 2.7, 3.0 and 3.3, as described in Example 8. FIG. 6A is a graph depicting a representative UV-trace profile of Protein L affinity chromatography using salt gradient elution from 100 mM NaCl to 0 mM at pH 2.4. FIG. 6B is a graph depicting a representative UV-trace profile of Protein L affinity chromatography using salt gradient elution from 100 mM NaCl to 0 mM at pH 2.7. FIG. 6C is a graph depicting a representative UV-trace profile of Protein L affinity chromatography using salt gradient elution from 100 mM NaCl to 0 mM at pH 3.0. FIG. 6D is a graph depicting a representative UV-trace profile of Protein L affinity chromatography using salt gradient elution from 100 mM NaCl to 0 mM at pH 3.3.

FIGS. 7A-7B illustrate separation of Ab #5 and Ab #6 by two-step purification in pH 2.7 and 3.0, as described in Example 9. FIG. 7A is a graph depicting a representative UV-trace profile of Protein L affinity chromatography using salt step elution at pH 2.7. A table summarizing the content of each peak is present. FIG. 7B is a graph depicting a representative UV-trace profile of Protein L affinity chromatography using salt step elution at pH 3.0. A table summarizing the content of each peak is present.

FIGS. 8A-8B illustrate separation of Ab #7 and Ab #8 by conductivity gradient and step in pH 3.0 as described in Example 10. FIG. 8A is a graph depicting a representative UV-trace profile of Protein L affinity chromatography using salt gradient elution from 100 mM NaCl to 0 mM at pH 3.0. FIG. 8B is a graph depicting a representative UV-trace profile of Protein L affinity chromatography using salt step elution at pH 3.0. A table summarizing the content of each peak is present.

FIGS. 9A-9C illustrate separation of Ab #8 and Ab #9 by conductivity gradient and step in pH 3.0 as described in Example 11. FIG. 9A is a graph depicting a representative UV-trace profile of Protein L affinity chromatography using salt gradient elution from 100 mM NaCl to 0 mM at pH 3.0. FIG. 9B is a graph depicting a representative UV-trace profile of Protein L affinity chromatography using salt step elution at pH 3.0. FIG. 9C is a SDS-PAGE image of protein samples derived from fractions in peak 1 and peak 2 shown in FIG. 9B which were analysed under non-reducing condition. MWM indicates the molecular weight marker. The gel was stained by coomassie brilliant blue.

FIGS. 10A-10B illustrate separation of monomeric and oligomeric BiTE antibodies (Ab #10) by conductivity gradient at pH 2.7, as described in Example 12. FIG. 10A is a graph depicting a representative UV-trace profile of Protein L affinity chromatography using salt gradient elution from 100 mM NaCl to 0 mM at pH 2.7. Fractions C7, C12, D5, D8, D11, E6, Ell, F4, and F9 were selected for SEC (size exclusion chromatography)-HPLC analysis. FIG. 10B shows a set of SEC-HPLC chromatograms of respective fractions from peak 1 and peak 2 shown in FIG. 10A. The analysis result of the molecular weight marker (MWM) is also shown in the lowest panel. The content of monomeric BiTE antibody (Ab #10) in each fraction is summarized in the right panel.

FIGS. 11A-11B illustrate separation of Ab #5 and Ab #6 by conductivity gradient and step in pH 3.0 using HiTrap Protein L column as described in Example 13. FIG. 11A is a graph depicting a representative UV-trace profile of Protein L affinity chromatography using salt gradient elution from 100 mM NaCl to 0 mM at pH 3.0. FIG. 11B is a graph depicting a representative UV-trace profile of Protein L affinity chromatography using salt step elution at pH 3.0. A table summarizing the content of each peak is present.

### DESCRIPTION OF EMBODIMENTS

The invention relates to, in part, methods of purifying a protein using Protein L. The invention also relates to, in part, methods of separating a protein using Protein L. The invention also relates to, in part, methods of isolating a protein using Protein L. The invention also relates to, in part, methods of producing a protein using Protein L.

In one aspect, the invention provides a method comprising the step of eluting a protein from a Protein L matrix by lowering a conductivity. In some embodiments, the protein comprises at least one Protein L binding motif. In some embodiments, at least two different proteins are eluted from the Protein L matrix, wherein each of the proteins comprises a different number of the Protein L binding motifs. In certain embodiments, the invention provides a method comprising the step of eluting at least two different proteins from a Protein L matrix by lowering a conductivity, wherein each of the proteins comprises a different number of Protein L binding motifs.

In another aspect, the method of the present invention further comprises the step of contacting a protein with a Protein L matrix. In some embodiments, the protein is bound to the Protein L matrix at a certain conductivity. In some embodiments, the protein may be comprised in a solution. In certain embodiments, the invention provides a method comprising the steps of (a) contacting a solution comprising at least two different proteins with a Protein L matrix at a certain conductivity so that the proteins are bound to the Protein L matrix, and (b) eluting the bound proteins from the Protein L matrix by lowering the conductivity, wherein each of the proteins comprises a different number of Protein L binding motifs.

The number of the Protein L binding motifs comprised in the protein can be one, two, three, four, five, six, seven, eight, nine, ten or more. In particular embodiments, the solution comprises two types of proteins, which are (i) a protein comprising one Protein L binding motif, and (ii) a protein comprising two Protein L binding motifs. Optionally, the solution may further comprise a protein comprising no Protein L binding motifs. In particular embodiments, the solution may comprise three types of proteins, which are (i) a protein comprising one Protein L binding motif, (ii) a protein comprising two Protein L binding motifs, and (iii) a protein comprising no Protein L binding motifs.

One of the possible effects of the present invention is that one protein can be separated from a mixture of at least two different proteins, each of which comprises a different number of the Protein L binding motifs. In the present invention, it can be determined that two proteins are separated when the elution positions of them are different and/or when the purity of them are increased as compared to before the purification, as described later. While not wishing to be bound by any particular theory, it can be speculated that the above effect would be based on the different binding affinities of the proteins to Protein L. In the present invention, a protein comprising a certain number of the Protein L binding motifs can be separated from proteins comprising a different number of the Protein L binding motifs. For example, a protein comprising one Protein L binding motif can be separated from proteins comprising two or more Protein L binding motifs, and optionally from a protein comprising no Protein L binding motifs.

In some embodiments, the Protein L binding motif described herein is an antibody kappa chain variable region. Any subgroup of kappa chain variable regions derived from any animal species can be used as a Protein L binding motif, as long as they have the binding ability to Protein L. In particular embodiments, the Protein L binding motif is selected from the group consisting of human variable kappa subgroup 1 (VK1, herein also described as V kappa 1), human variable kappa subgroup 3 (VK3, herein also described as V kappa 3), human variable kappa subgroup 4 (VK4, herein also described as V kappa 4), and mouse variable kappa subgroup 1 (VK1, herein also described as V kappa 1). In further embodiments, for example, human VK1 is selected from the group consisting of VK1-5, VK1-6, VK1-8, VK1-9, VK1-12, VK1-13, VK1-16, VK1-17, VK1-22, VK1-27, VK1-32, VK1-33, VK1-35, VK1-37, VK1-39, VK1D-8, VK1D-12, VK1D-13, VK1D-16, VK1D-17, VK1D-22, VK1D-27, VK1D-32, VK1D-33, VK1D-35, VK1D-37, VK1D-39, VK1D-42, VK1D-43, and VK1-NL1; human VK3 is selected from the group consisting of VK3-7, VK3-11, VK3-15, VK3-20, VK3-25, VK3-31, VK3-34, VK3D-7, VK3D-11, VK3D-15, VK3D-20, VK3D-25, VK3D-31, and VK3D-34; human VK4 is selected from the group consisting of VK4-1; and mouse VK1 is selected from the group consisting of VK1-35, VK1-88, VK1-99, VK1-108, VK1-110, VK1-115, VK1-117, VK1-122, VK1-131, VK1-132, VK1-133, VK1-135, and VK1-136. Specific examples of the amino acid sequences of the above-described antibody kappa chain variable regions can be found, for example, in Kabat et al., Sequences of Proteins of Immunological Interest, 5th Ed. Public Health Service, National Institutes of Health, Bethesda, Md., 1991. In certain embodiments, variants of the above kappa chain variable region which have amino acid modifications are also included in the Protein L binding motif as long as they still have the binding ability to Protein L. A fragment of the above kappa chain variable region can also be included in the Protein L binding motif as long as it still has the binding ability to Protein L. In the prior art, some VL amino acid residues involved in the interaction with Protein L have already been identified (see, e.g., Graille et al (2002), Structure 9(8): 679-687). Referring to such information, a skilled person would be able to design and prepare a fragment of the antibody kappa chain variable region which has the binding ability to Protein L without undue burden.

On the other hand, light chain variable regions which do not bind to Protein L are defined as a Protein L non-binding motif in the present invention. Any subgroup of light chain variable regions derived from any animal species can be used as a Protein L non-binding motif, as long as they have no binding ability to Protein L. For example, the following light chain variable regions are classified into the Protein L non-binding motif: human variable kappa subgroup 2 (VK2, herein also described as V kappa 2), any subgroup of human variable lambda, and any subgroup of mouse variable lambda. In further embodiments, for example, human VK2 is selected from the group consisting of VK2-4, VK2-10, VK2-14, VK2-18, VK2-19, VK2-23, VK2-24, VK2-26, VK2-28, VK2-29, VK2-30, VK2-36, VK2-38, VK2-40, VK2D-10, VK2D-14, VK2D-18, VK2D-19, VK2D-23, VK2D-24, VK2D-26, VK2D-28, VK2D-29, VK2D-30, VK2D-36, VK2D-38, and VK2D-40; human variable lambda is selected from the group consisting of VL1-36, VL1-40, VL1-41, VL1-44, VL1-47, VL1-50, VL1-51, VL1-62, VL2-5, VL2-8, VL2-11, VL2-14, VL2-18, VL2-23, VL2-28, VL2-33, VL2-34, VL3-1, VL3-2, VL3-4, VL3-6, VL3-7, VL3-9, VL3-10, VL3-12, VL3-13, VL3-15, VL3-16, VL3-17, VL3-19, VL3-21, VL3-22, VL3-24, VL3-25, VL3-26, VL3-27, VL3-29, VL3-30, VL3-31, VL3-32, VL4-3, VL4-60, and VL4-69; mouse variable lambda is selected from the group consisting of VL1, VL2, VL3, VL4, and VL8. Specific examples of the amino acid sequences of the above-described light chain variable regions can be found, for example, in Kabat et al., Sequences of Proteins of Immunological Interest, 5th Ed. Public Health Service, National Institutes of Health, Bethesda, Md., 1991. In certain embodiments, variants of the above light chain variable region which have amino acid modifications are also included in the Protein L non-binding motif as long as they still have no binding ability to Protein L. In other embodiments, variants of a Protein L binding motif (such as, for example, human VK1, human VK3, human VK4, or mouse VK1) which have amino acid modifications to lose the binding ability to Protein L are also included in the Protein L non-binding motif. For example, S12P mutant of human VK1, wherein Ser at position 12 is substituted with Pro (numbered according to the Kabat numbering system), is an example of the Protein L non-binding motif. A fragment of the above light chain variable region can also be included in the Protein L non-binding motif as long as it still has no binding ability to Protein L.

A protein comprising at least one Protein L binding motif described herein can be a monomeric protein which comprises only a single polypeptide, or a multimeric protein which comprises two or more polypeptides. The multimeric protein can be a homomultimeric protein or a heteromultimeric protein. In the case of a heteromultimeric protein which comprises at least two different polypeptides, each of the polypeptides can comprise any number of the Protein L binding motifs or can comprise no Protein L binding motifs, as long as at least one Protein L binding motif is comprised in the protein. In particular embodiments, a heteromultimeric protein comprises two different polypeptides, one of which comprises one Protein L binding motif and the other of which comprises no Protein L binding motifs. In the case of a homomultimeric protein which comprises at least two identical polypeptides, each of the polypeptides can comprise any number of the Protein L binding motifs, as long as at least two Protein L binding motifs are comprised in the protein. In particular embodiments, a homomultimeric protein comprises two identical polypeptides, both of which comprise one Protein L binding motif.

In some embodiments, the protein comprising at least one Protein L binding motif described herein is an antibody. The term “antibody” herein is used in the broadest sense and encompasses various antibody structures, including but not limited to monoclonal antibodies, polyclonal antibodies, monospecific antibodies, and multispecific antibodies (e.g., bispecific antibodies), so long as they exhibit the desired antigen-binding activity. The antibody can be a whole antibody or an antibody fragment. In general, multispecific antibodies comprise multiple antigen-binding domains derived from two or more different antibodies. The epitopes of a multispecific antibody can be located on multiple antigens or on a single antigen. Bispecific antibodies can comprise, for example, a combination of two different light chains and two different heavy chains. Alternatively, bispecific antibodies can comprise a combination of two different light chains and one common heavy chain, or a combination of one common light chain and two different heavy chains. In other embodiments, antibodies in artificially-modified formats such as, for example, CrossMab, CrossMab-Fab, Dual Action Fab (DAF), DutaMab, LUZ-Y, SEEDbody, DuoBody, kappa-lambda body, Dual Variable Domain Immunoglobulin (DVD-Ig), scFab-IgG, Fab-scFab-IgG, IgG-scFv, and IgG-Fab (see, e.g., Spiess et al. (2015) Mol Immunol 67:95-106, Brinkmann U et al. (2017) MAbs 9(2):182-212), are also included in the term of “antibody”, so long as they have the desired antigen-binding activity. In other embodiments, antibody derivatives such as an antibody fused with one or more other polypeptides, or an antibody conjugated with one or more other agents (e.g., drugs, toxins, radioisotopes, and polymers) are also included in the term of “antibody”, so long as they have the desired antigen-binding activity.

The terms “full length antibody,” “intact antibody,” and “whole antibody” are used herein interchangeably to refer to an antibody having a structure substantially similar to a naturally occurring immunoglobulin structure. For example, a native IgG molecule is a heterotetrameric glycoprotein of about 150,000 daltons, composed of two identical light chains and two identical heavy chains that are disulfide-bonded. From N- to C-terminus, each light chain has a variable domain (VL), followed by a constant domain (CL). Similarly, from N- to C-terminus, each heavy chain has a variable domain (VH), followed by three constant domains (CH1, CH2, and CH3). The antibody described herein can be of any class and any subclass (for example, IgG1, IgG2, IgG3, IgG4, IgA1, IgA2, IgD, IgE, and IgM). The heavy chain constant domain of the antibody can be derived from IgA (alpha), IgD (delta), IgE (epsilon), IgG (gamma), or IgM (mu). The light chain of the antibody can be kappa or lambda. Antibodies can be made by various techniques, including but not limited to immunization of animals against an antigen as well as production by recombinant host cells as described below. See also e.g., U.S. Pat. No. 4,816,567.

An “antibody fragment” refers to a molecule other than a whole antibody that comprises a portion of a whole antibody that binds to the antigen to which the whole antibody binds. Examples of antibody fragments include but are not limited to Fab, Fab′, Fab′-SH, F(ab′)2, Fv, single-domain antibody (sdAb), single-chain Fv (scFv), diabodies, scFv dimers, tandem scFv (taFv), (scFv)2, single-chain diabodies (scDb), single-chain Fab (scFab), tandem scDb (TandAb), triabodies, tetrabodies, hexabodies, one-armed antibodies, and multispecific antibodies formed from antibody fragments such as Fab-scFv, scFv-Fc, Fab-scFv-Fc, scDb-Fc, and taFv-Fc. Antibody fragments can be made by various techniques, including but not limited to proteolytic digestion of a whole antibody as well as production by recombinant host cells as described below. For a review of certain antibody fragments, see, e.g., Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO1993/16185; and U.S. Pat. Nos. 5,571,894 and 5,587,458. For discussion of Fab and F(ab′)2 fragments, see, e.g., U.S. Pat. No. 5,869,046.

Diabodies are antibody fragments with two antigen-binding sites that may be bivalent or bispecific. See, for example, EP 404,097; WO1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

One-armed antibodies are described in, for example, WO2005/063816; Martens et al, Clin Cancer Res (2006), 12: 6144. For treatment of pathological conditions requiring an antagonistic function, and where bivalency of an antibody results in an undesirable agonistic effect, the monovalent trait of a one-armed antibody (i.e., an antibody comprising a single antigen binding domain) results in and/or ensures an antagonistic function upon binding of the antibody to a target molecule. Furthermore, the one-armed antibody comprising an Fc region is characterized by superior pharmacokinetic attributes (such as an enhanced half life and/or reduced clearance rate in vivo) compared to Fab forms having similar/substantially identical antigen binding characteristics, thus overcoming a major drawback in the use of conventional monovalent Fab antibodies. Techniques for making one-armed antibodies include, but are not limited to, “knobs-in-holes” engineering (see, e.g., U.S. Pat. No. 5,731,168).

Techniques for making multispecific antibodies include, but are not limited to, recombinant co-expression of two immunoglobulin heavy chain and light chain pairs having different specificities (see Milstein and Cuello, Nature 305: 537 (1983)), WO1993/08829, and Traunecker et al., EMBO J. 10: 3655 (1991)). One of the major obstacles in the development of bispecific antibodies has been the difficulty of producing the material in sufficient quality and quantity by traditional technologies, such as the hybrid hybridoma and chemical conjugation methods. Co-expression of two antibodies, consisting of different heavy and light chains, in a host cell leads to a mixture of possible antibody byproducts in addition to the desired bispecific antibody.

Numerous multispecific antibody formats have been developed in the art to address therapeutic opportunities afforded by molecules with multiple binding specificities. Several approaches have been described to prepare bispecific antibodies in which specific antibody light chains or fragments pair with specific antibody heavy chains or fragments.

For example, knobs-into-holes is a heterodimerization technology for the CH3 domain of an antibody. Previously, knobs-into-holes technology has been applied to the production of human full-length bispecific antibodies with a single common light chain (LC) (Merchant et al. (1998) Nat Biotechnol. 16: 677-681; Jackman et al. (2010) J Biol Chem. 285: 20850-20859; WO1996/027011).

Schaefer et al. describe a method to assemble two heavy and two light chains, derived from two existing antibodies, into bispecific antibodies without use of artificial linkers (PNAS (2011) 108(27): 11187-11192 and US2009/0232811). Based on the knobs-into-holes technology that enables heterodimerization of the heavy chains, correct association of the light chains and their cognate heavy chains is achieved by exchange of heavy chain and light chain domains within the Fab of one half of the bispecific antibody (CrossMab). This “crossover” retains the antigen-binding affinity but makes the two arms so different that light-chain mispairing can no longer occur (see, e.g., WO2009/080251, WO2009/080252, WO2009/080253, and WO2009/080254).

International Patent Publication No. WO2011/131746 describes an in vitro method for generating a bispecific antibody in which asymmetrical mutations are introduced into the CH3 regions of two monospecific starting antibodies in order to drive directional “Fab-arm” or “half-molecule” exchange between two monospecific IgG4 or IgG4-like antibodies upon incubation under reducing conditions. Strop et al. describe a method of producing stable bispecific antibodies by expressing and purifying two antibodies of interest separately, and then mixing them together under specified redox conditions (J Mol Biol. (2012) 420: 204-219).

Other heterodimerization domain having a strong preference for forming heterodimers over homodimers can be incorporated into the multispecific antibody formats. Illustrative examples include but are not limited to, for example, WO2007/147901 (Kjaergaard et al., describing ionic interactions); WO2009/089004 (Kannan et al., describing electrostatic steering effects); WO2010/034605 (Christensen et al., describing coiled coils).

Zhu et al. have engineered mutations in the VL/VH interface of a diabody construct consisting of variable domain antibody fragments completely devoid of constant domains, and generated a heterodimeric diabody (Protein Science (1997) 6:781-788). Similarly, Igawa et al. have also engineered mutations in the VL/VH interface of a single-chain diabody to promote selective expression and inhibit conformational isomerization of the diabody (Protein Engineering, Design & Selection (2010) 23:667-677).

Another format, used for Bispecific T cell Engager (BiTE) molecules (see, e.g., Wolf et al. (2005) Drug Discovery Today 10:1237-1244), is based on scFv modules. A BiTE concatenates two scFv fragments of different specificities in tandem on a single chain. This configuration precludes the production of molecules with two copies of the same heavy chain variable region. In addition, the linker configuration is designed to ensure correct pairing of the respective light and heavy chains.

Any antibody molecules in any format which comprise at least one antibody kappa chain variable region described above can be used as a protein comprising at least one Protein L binding motif described herein.

The antibody described herein can comprise any types of light chain constant regions derived from any animal species. Regarding the two classes of light chains (kappa and lambda), the variable region and the constant region can belong to the same class, or classes different from each other. For example, a light chain may comprise a combination of a kappa variable region and a kappa constant region. Alternatively, a light chain may comprise a combination of a kappa variable region and a lambda constant region. In addition, the variable region and the constant region can be derived from the same animal species, or animal species different from each other. For example, a light chain may comprise a combination of a human-derived variable region and a human-derived constant region. Alternatively, a light chain may comprise a combination of a mouse-derived variable region and a human-derived constant region. In certain embodiments, a human kappa constant region has an amino acid sequence of SEQ ID NO: 1, and a human lambda constant region has an amino acid sequence of SEQ ID NO: 2.

In the present invention, an antibody comprising a light chain, which comprises a kappa variable region and a lambda constant region is provided. In the present invention, an antibody comprising a light chain, which comprises a lambda variable region and a kappa constant region is also provided. In further embodiments, the antibody comprise another light chain, which comprises any one of (i) a kappa variable region and a kappa constant region, (ii) a lambda variable region and a lambda constant region, (iii) a kappa variable region and a lambda constant region, or (iv) a lambda variable region and a kappa constant region. In the present invention, an antibody is provided, which comprises two light chains, one of which comprises a kappa variable region and a lambda constant region, and the other of which comprises any one of (i) a kappa variable region and a kappa constant region, (ii) a lambda variable region and a lambda constant region, (iii) a kappa variable region and a lambda constant region, or (iv) a lambda variable region and a kappa constant region. In the present invention, an antibody is also provided, which comprises two light chains, one of which comprises a lambda variable region and a kappa constant region, and the other of which comprises any one of (i) a kappa variable region and a kappa constant region, (ii) a lambda variable region and a lambda constant region, (iii) a kappa variable region and a lambda constant region, or (iv) a lambda variable region and a kappa constant region. The antibody can be a monospecific antibody or a multispecific (e.g., bispecific) antibody. Alternatively, the antibody can be an antibody fragment such as, for example, Fab, Fab′, Fab′-SH, F(ab′)2, single-chain Fab (scFab), and one-armed antibodies. In certain embodiments, the antibody comprises a Protein L binding motifs as a kappa variable region.

In some embodiments, an antibody described herein comprises two light chains, one of which comprises one Protein L binding motif. In further embodiments, the other of the two light chains of the antibody comprises one Protein L non-binding motif. In further embodiments, two heavy chains of the antibody can be identical or non-identical. In certain embodiments, the antibody can be a monospecific antibody or a multispecific (e.g., bispecific) antibody. A monospecific antibody usually comprises two identical light chains and two identical heavy chains. A bispecific antibody usually comprises two different light chains and two different heavy chains. Alternatively, a bispecific antibody can comprises two different light chains and one common heavy chain, or one common light chain and two different heavy chains.

In the present invention, both an antibody comprising one Protein L binding motif (referred to as antibody A) and an antibody comprising two Protein L binding motifs (referred to as antibody B) can be present in a solution as a mixture. By applying a method of the present invention to such a mixture, separation of the antibody A from the antibody B can be expected. In certain embodiments, the antibody A can be an antibody comprising two light chains, one of which comprises a Protein L binding motif, and the other of which comprises a Protein L non-binding motif. In certain embodiments, the antibody B can be an antibody comprising two light chains, both of which comprise a Protein L binding motif. In particular embodiments, the solution can comprise two types of antibodies, which are (i) an antibody comprising two light chains, one of which comprises a Protein L binding motif, and the other of which comprises a Protein L non-binding motif, and (ii) an antibody comprising two light chains, both of which comprise a Protein L binding motif. In another embodiment, the solution can comprise two types of antibodies, which are (i) an antibody comprising two light chains, one of which comprises a Protein L binding motif, and the other of which comprises a Protein L non-binding motif, and (ii) an antibody comprising two light chains, both of which are the same as the light chain of the antibody described in (i) which comprises a Protein L binding motif. In further embodiments, the antibody described in (i) is a bispecific antibody, and the antibody described in (ii) is a monospecific antibody. In further embodiments, one of the two heavy chains of the antibody described in (i) is the same as the heavy chain of the antibody described in (ii). In further embodiments, one of the two pairs of the heavy and light chains of the antibody described in (i) is the same as the pair of the heavy and light chains of the antibody described in (ii). In the present invention, the antibodies described in (i) and (ii) can work as a protein comprising one Protein L binding motif and a protein comprising two Protein L binding motifs, respectively. By applying a method of the present invention to the mixture of the above two antibodies, separation of the antibody described in (i) from the antibody described in (ii) can be expected.

Optionally, the solution can comprise three types of antibodies, which are (i) an antibody comprising two light chains, one of which comprises a Protein L binding motif, and the other of which comprises a Protein L non-binding motif, and (ii) an antibody comprising two light chains, both of which comprise a Protein L binding motif, and (iii) an antibody comprising two light chains, both of which comprise a Protein L non-binding motif. In another embodiment, the solution can comprise three types of antibodies, which are (i) an antibody comprising two light chains, one of which comprises a Protein L binding motif, and the other of which comprises a Protein L non-binding motif, (ii) an antibody comprising two light chains, both of which are the same as the light chain of the antibody described in (i) which comprises a Protein L binding motif, and (iii) an antibody comprising two light chains, both of which are the same as the light chain of the antibody described in (i) which comprises a Protein L non-binding motif. In further embodiments, the antibody described in (i) is a bispecific antibody, and the antibodies described in (ii) and (iii) are monospecific antibodies. In further embodiments, one of the two heavy chains of the antibody described in (i) is the same as the heavy chain of the antibody described in (ii), and the other of the two heavy chains of the antibody described in (i) is the same as the heavy chain of the antibody described in (iii). In further embodiments, one of the two pairs of the heavy and light chains of the antibody described in (i) is the same as the pair of the heavy and light chains of the antibody described in (ii), and the other of the two pairs of the heavy and light chains of the antibody described in (i) is the same as the pair of the heavy and light chains of the antibody described in (iii). In the present invention, the antibodies described in (i), (ii), and (iii) can work as a protein comprising one Protein L binding motif, a protein comprising two Protein L binding motifs, and a protein comprising no Protein L binding motifs, respectively. By applying a method of the present invention to the mixture of the above three antibodies, separation of the antibody described in (i) from the antibodies described in (ii) and (iii) can be expected.

In some embodiments, a one-armed antibody described herein comprises only one light chain, which comprises a Protein L binding motif. A one-armed antibody usually comprises one light chain, one heavy chain, and one heavy chain Fc region.

In the present invention, both an antibody comprising one Protein L binding motif (referred to as antibody A) and an antibody comprising two Protein L binding motifs (referred to as antibody B) can be present in a solution as a mixture. By applying a method of the present invention to such a mixture, separation of the antibody A from the antibody B can be expected. In certain embodiments, the antibody A can be an antibody comprising only one light chain which comprises a Protein L binding motif. In certain embodiments, the antibody B can be an antibody comprising two light chains, both of which comprise a Protein L binding motif. In particular embodiments, the solution can comprise two types of antibodies, which are (i) an antibody comprising only one light chain which comprises a Protein L binding motif, and (ii) an antibody comprising two light chains, both of which comprise a Protein L binding motif. In another embodiment, the solution can comprise two types of antibodies, which are (i) an antibody comprising only one light chain which comprises a Protein L binding motif, and (ii) an antibody comprising two light chains, both of which are the same as the light chain of the antibody described in (i) which comprises a Protein L binding motif. In further embodiments, the antibody described in (i) is a one-armed antibody, and the antibody described in (ii) is a whole antibody. In further embodiments, the heavy chain of the antibody described in (i) is the same as the heavy chain of the antibody described in (ii). In further embodiments, the pair of the heavy and light chains of the antibody described in (i) is the same as the pair of the heavy and light chains of the antibody described in (ii). In the present invention, the antibodies described in (i) and (ii) can work as a protein comprising one Protein L binding motif and a protein comprising two Protein L binding motifs, respectively. By applying a method of the present invention to the mixture of the above two antibodies, separation of the antibody described in (i) from the antibody described in (ii) can be expected.

Optionally, the solution can comprise three types of proteins, which are (i) an antibody comprising only one light chain which comprises a Protein L binding motif, (ii) an antibody comprising two light chains, both of which comprise a Protein L binding motif, and (iii) a dimeric protein comprising two heavy chain Fc regions. In another embodiment, the solution can comprise three types of proteins, which are (i) an antibody comprising only one light chain which comprises a Protein L binding motif, (ii) an antibody comprising two light chains, both of which are the same as the light chain of the antibody described in (i) which comprises a Protein L binding motif, and (iii) a dimeric protein comprising two heavy chain Fc regions. In further embodiments, the antibody described in (i) is a one-armed antibody, and the antibody described in (ii) is a whole antibody. In further embodiments, the heavy chain of the antibody described in (i) is the same as the heavy chain of the antibody described in (ii). In further embodiments, the pair of the heavy and light chains of the antibody described in (i) is the same as the pair of the heavy and light chains of the antibody described in (ii). In the present invention, the proteins described in (i), (ii), and (iii) can work as a protein comprising one Protein L binding motif, a protein comprising two Protein L binding motifs, and a protein comprising no Protein L binding motifs, respectively. By applying a method of the present invention to the mixture of the above three proteins, separation of the antibody described in (i) from the proteins described in (ii) and (iii) can be expected.

In some embodiments, an antibody fragment such as, for example, a single-chain Fv (scFv), diabody, scFv dimer, tandem scFv (taFv), (scFv)2, single-chain diabody (scDb), single-chain Fab (scFab), tandem scDb (TandAb), triabody, and tetrabody described herein comprises at least one Protein L binding motif. In further embodiments, the antibody fragment may additionally comprise at least one Protein L non-binding motif. For example, a scFv usually comprises one light chain variable region and one heavy chain variable region. For example, a diabody, scFv dimer, taFv, (scFv)2, scDb, and scFab usually comprise two light chain variable regions and two heavy chain variable regions. For example, a triabody usually comprises three light chain variable regions and three heavy chain variable regions. For example, a TandAb and tetrabody usually comprise four light chain variable regions and four heavy chain variable regions.

In the present invention, both an antibody fragment comprising at least one Protein L binding motif and a multimer (e.g., dimer) thereof can be present in a solution as a mixture. In general, single-chain antibody fragments such as scFv, diabody, scFv dimer, taFv, (scFv)2, scDb, scFab, TandAb, triabody, and tetrabody have a tendency to associate into multimers (e.g., dimers) through the interactions between, for example, a VH domain existing on one fragment and a VL domain existing on another fragment. By applying a method of the present invention to such a mixture, separation of the antibody fragment from the multimer (e.g., dimer) thereof can be expected. In certain embodiments, the solution can comprise two types of proteins, which are (i) an antibody fragment comprising at least one Protein L binding motif, and (ii) a multimer (e.g., dimer) of the antibody fragment described in (i). In further embodiments, the antibody fragment described in (i) is any one of scFv, diabody, scFv dimer, taFv, (scFv)2, scDb, scFab, TandAb, triabody, and tetrabody. In the present invention, the proteins described in (i) and (ii) can work as a protein comprising at least one Protein L binding motif and a protein comprising at least two Protein L binding motifs, respectively. By applying a method of the present invention to the mixture of the above two proteins, separation of the antibody fragment described in (i) from the multimer (e.g., dimer) thereof described in (ii) can be expected.

In the present invention, isolated nucleic acid encoding a protein comprising at least one Protein L binding motif is provided. The present invention also provides one or more vectors (e.g., expression vectors) comprising such nucleic acid. The present invention also provides a host cell comprising such nucleic acid. In one embodiment, a host cell comprises (e.g., has been transformed with): (1) a vector comprising a first nucleic acid that encodes a light chain of an antibody and a second nucleic acid that encodes a heavy chain of the antibody, or (2) a first vector comprising a nucleic acid that encodes a light chain of an antibody and a second vector comprising a nucleic acid that encodes a heavy chain of the antibody. In another embodiment, a host cell comprises one or more vectors (e.g., expression vectors) comprising more than two nucleic acids that encode light and heavy chains of a multispecific antibody. The term “host cell” used herein refers to cells into which exogenous nucleic acid has been introduced, including the progeny of such cells. The present invention also provides a method of making a protein comprising at least one Protein L binding motif, wherein the method comprises culturing a host cell comprising a nucleic acid encoding the protein, under conditions suitable for expression of the protein, and optionally collecting the protein from the host cell (or host cell culture medium). Antibodies may be produced using recombinant methods and compositions, e.g., as described in U.S. Pat. No. 4,816,567.

For recombinant production of a protein comprising at least one Protein L binding motif described herein, nucleic acid encoding the protein is isolated and inserted into one or more vectors for further cloning and/or expression in a host cell. Such nucleic acid may be readily isolated and sequenced using conventional procedures (e.g., by using oligonucleotide probes that are capable of binding specifically to nucleic acids of interest).

Suitable host cells for cloning or expression of vectors include prokaryotic or eukaryotic cells. For example, proteins may be produced in bacteria, in particular when glycosylation are not needed. For expression of antibody fragments in bacteria, see, e.g., U.S. Pat. Nos. 5,648,237, 5,789,199, and 5,840,523 (see also Charlton, Methods in Molecular Biology, Vol. 248 (B. K. C. Lo, ed., Humana Press, Totowa, N.J., 2003), pp. 245-254, describing expression of antibody fragments in E. coli). After expression, the protein may be isolated from the bacterial cell paste in a soluble fraction and can be further purified. In addition to prokaryotes, eukaryotic cells such as fungi, yeast, plant, insect or mammalian cells are also suitable hosts for cloning or expression of glycosylated protein. Examples of useful mammalian cell lines are COS7, 293, BHK, CV1, VERO76, HeLa, MDCK, BRL3A, W138, HepG2, MMT060562, TRI, MRC5, FS4, CHO, Y0, NS0, and Sp2/0 cells. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In another aspect, the method of the present invention further comprises the step of collecting a protein eluted from the Protein L matrix. In certain embodiments, the invention provides a method comprising the steps of (a) eluting at least two different proteins from a Protein L matrix by lowering a conductivity, and (b) collecting one of the eluted proteins, wherein each of the proteins comprises a different number of Protein L binding motifs. In certain embodiments, the invention provides a method comprising the steps of (a) contacting a solution comprising at least two different proteins with a Protein L matrix at a certain conductivity so that the proteins are bound to the Protein L matrix, (b) eluting the bound proteins from the Protein L matrix by lowering the conductivity, and (c) collecting one of the eluted proteins, wherein each of the proteins comprises a different number of Protein L binding motifs.

In another aspect, the method of the present invention further comprises the steps of (a) culturing a cell which expresses a protein and (b) collecting the protein. In some embodiments, the cell expresses one or more types of proteins when cultured under suitable conditions. In some embodiments, any one of the proteins is expressed inside of the cell or secreted into the cell culture medium. In some embodiments, the expressed protein is collected from the cell or cell culture medium. Any kind of cells can be used as long as they express the protein, such as native cells, transformed cells with exogenous nucleic acid, and fused cells such as hybridomas and hybrid hybridomas (quadromas). A single type of cell or a mixture of two or more types of cells can be cultured. In certain embodiments, the invention provides a method comprising the steps of (a) culturing cells under conditions suitable for expression of at least two different proteins, (b) collecting a solution comprising the proteins expressed in the cells, (c) contacting the solution with a Protein L matrix at a certain conductivity so that the proteins are bound to the Protein L matrix, and (d) eluting the bound proteins from the Protein L matrix by lowering the conductivity, wherein each of the proteins comprises a different number of Protein L binding motifs.

In further embodiments, a polypeptide comprises at least one Protein L binding motif. In further embodiments, at least two different proteins are formed, each of which comprises a different number of the polypeptide. In certain embodiments, the invention provides a method comprising the steps of (a) culturing cells under conditions suitable for expression of a polypeptide comprising at least one Protein L binding motif, (b) collecting a solution comprising at least two different proteins expressed in the cells, wherein each of the proteins comprises a different number of the polypeptide, (c) contacting the solution with a Protein L matrix at a certain conductivity so that the proteins are bound to the Protein L matrix, and (d) eluting the bound proteins from the Protein L matrix by lowering the conductivity.

In another aspect, the method of the present invention further comprises the steps of (a) isolating a nucleic acid and (b) transforming host cells with the nucleic acid. In some embodiments, the nucleic acid encodes a polypeptide comprising at least one Protein L binding motif. In some embodiments, the nucleic acid is inserted into one or more expression vectors. In certain embodiments, the invention provides a method comprising the steps of (a) isolating a nucleic acid which encodes a polypeptide comprising at least one Protein L binding motif, (b) transforming host cells with an expression vector comprising the nucleic acid, (c) culturing the host cells under conditions suitable for expression of the polypeptide, (d) collecting a solution comprising at least two different proteins expressed in the host cells, wherein each of the proteins comprises a different number of the polypeptides, (e) contacting the solution with a Protein L matrix at a certain conductivity so that the proteins are bound to the Protein L matrix, and (f) eluting the bound proteins from the Protein L matrix by lowering the conductivity.

Hereinafter a polypeptide comprising at least one Protein L binding motif is referred to as a polypeptide A, and a polypeptide comprising no Protein L binding motifs is referred to as a polypeptide B. In certain embodiments, at least three different multimeric (e.g., dimeric) proteins are formed, each of which comprises a different combination of the polypeptide A and polypeptide B. In particular embodiments, the invention provides a method comprising the steps of (a) culturing cells under conditions suitable for expression of a polypeptide A and polypeptide B, (b) collecting a solution comprising at least three types of proteins expressed in the cells, which are (i) a heteromultimeric (e.g., heterodimeric) protein comprising at least one polypeptide A and at least one polypeptide B, (ii) a homomultimeric (e.g., homodimeric) protein comprising at least two polypeptides A, and (iii) a homomultimeric (e.g., homodimeric) protein comprising at least two polypeptides B, (c) contacting the solution with a Protein L matrix at a certain conductivity so that the proteins comprising at least one polypeptide A are bound to the Protein L matrix, and (d) eluting the bound proteins from the Protein L matrix by lowering the conductivity.

In particular embodiments, the invention provides a method comprising the steps of (a) isolating a nucleic acid which encodes a polypeptide A and a nucleic acid which encodes a polypeptide B, (b) transforming host cells with one or more expression vectors comprising the nucleic acids, (c) culturing the host cells under conditions suitable for expression of the polypeptide A and polypeptide B, (d) collecting a solution comprising at least three types of proteins expressed in the host cells, which are (i) a heteromultimeric (e.g., heterodimeric) protein comprising at least one polypeptide A and at least one polypeptide B, (ii) a homomultimeric (e.g., homodimeric) protein comprising at least two polypeptides A, and (iii) a homomultimeric (e.g., homodimeric) protein comprising at least two polypeptides B, (e) contacting the solution with a Protein L matrix at a certain conductivity so that the proteins comprising at least one polypeptide A are bound to the Protein L matrix, and (f) eluting the bound proteins from the Protein L matrix by lowering the conductivity.

In some embodiments, a protein comprising at least one Protein L binding motif in the present invention is an antibody. In certain embodiments, the antibody comprises two light chains, one of which comprises one Protein L binding motif (referred to as a light chain A), and the other of which comprises one Protein L non-binding motif (referred to as a light chain B). In certain embodiments, three types of antibodies are formed, each of which comprises a different combination of the light chain A and the light chain B. In particular embodiments, the invention provides a method comprising the steps of (a) culturing cells under conditions suitable for expression of a light chain A, a light chain B, and one or more heavy chains, (b) collecting a solution comprising three types of antibodies expressed in the cells, which are (i) an antibody comprising one light chain A and one light chain B, (ii) an antibody comprising two light chains A, and (iii) an antibody comprising two light chains B, (c) contacting the solution with a Protein L matrix at a certain conductivity so that the antibodies comprising at least one light chain A are bound to the Protein L matrix, and (d) eluting the bound antibodies from the Protein L matrix by lowering the conductivity. In further embodiments, the antibody described in (i) is a bispecific antibody, and the antibodies described in (ii) and (iii) are monospecific antibodies.

In particular embodiments, the invention provides a method comprising the steps of (a) isolating a nucleic acid which encodes a light chain A, a nucleic acid which encodes a light chain B, and nucleic acids which encode one or more heavy chains, (b) transforming host cells with one or more expression vectors comprising the nucleic acids, (c) culturing the host cells under conditions suitable for expression of the light chain A, light chain B, and heavy chains, (d) collecting a solution comprising three types of antibodies expressed in the host cells, which are (i) an antibody comprising one light chain A and one light chain B, (ii) an antibody comprising two light chains A, and (iii) an antibody comprising two light chains B, (e) contacting the solution with a Protein L matrix at a certain conductivity so that the antibodies comprising at least one light chain A are bound to the Protein L matrix, and (f) eluting the bound antibodies from the Protein L matrix by lowering the conductivity. In further embodiments, the antibody described in (i) is a bispecific antibody, and the antibodies described in (ii) and (iii) are monospecific antibodies.

In certain embodiments, at least three different multimeric (e.g., dimeric) proteins are formed, each of which comprises a different number of the polypeptides A. In particular embodiments, the invention provides a method comprising the steps of (a) culturing cells under conditions suitable for expression of a polypeptide A, (b) collecting a solution comprising at least three types of proteins expressed in the cells, which are (i) a heteromultimeric (e.g., heterodimeric) protein comprising at least one polypeptide A, (ii) a homomultimeric (e.g., homodimeric) protein comprising at least two polypeptides A, and (iii) a homomultimeric (e.g., homodimeric) protein comprising no polypeptides A, (c) contacting the solution with a Protein L matrix at a certain conductivity so that the proteins comprising at least one polypeptide A are bound to the Protein L matrix, and (d) eluting the bound proteins from the Protein L matrix by lowering the conductivity.

In particular embodiments, the invention provides a method comprising the steps of (a) isolating a nucleic acid which encodes a polypeptide A, (b) transforming host cells with an expression vector comprising the nucleic acid, (c) culturing the host cells under conditions suitable for expression of the polypeptide A, (d) collecting a solution comprising at least three types of proteins expressed in the host cells, which are (i) a heteromultimeric (e.g., heterodimeric) protein comprising at least one polypeptide A, (ii) a homomultimeric (e.g., homodimeric) protein comprising at least two polypeptides A, and (iii) a homomultimeric (e.g., homodimeric) protein comprising no polypeptides A, (e) contacting the solution with a Protein L matrix at a certain conductivity so that the proteins comprising at least one polypeptide A are bound to the Protein L matrix, and (f) eluting the bound proteins from the Protein L matrix by lowering the conductivity.

In some embodiments, a protein comprising at least one Protein L binding motif in the present invention is a one-armed antibody. In certain embodiments, the antibody comprises only one light chain, which comprises one Protein L binding motif (referred to as a light chain A). In certain embodiments, three different proteins are formed, each of which comprises a different number of the light chain A. In particular embodiments, the invention provides a method comprising the steps of (a) culturing cells under conditions suitable for expression of a light chain A, a heavy chain, and a heavy chain Fc region, (b) collecting a solution comprising three types of proteins expressed in the cells, which are (i) an antibody comprising only one light chain A, (ii) an antibody comprising two light chains A, and (iii) a dimeric protein comprising two heavy chain Fc regions, (c) contacting the solution with a Protein L matrix at a certain conductivity so that the antibodies comprising at least one light chain A are bound to the Protein L matrix, and (d) eluting the bound antibodies from the Protein L matrix by lowering the conductivity. In further embodiments, the antibody described in (i) is a one-armed antibody, and the antibody described in (ii) is a whole antibodies.

In particular embodiments, the invention provides a method comprising the steps of (a) isolating a nucleic acid which encodes a light chain A, a nucleic acid which encodes a heavy chain, and a nucleic acid which encodes a heavy chain Fc region, (b) transforming host cells with one or more expression vectors comprising the nucleic acids, (c) culturing the host cells under conditions suitable for expression of the light chain A, heavy chain, and heavy chain Fc region, (d) collecting a solution comprising three types of proteins expressed in the host cells, which are (i) an antibody comprising only one light chain A, (ii) an antibody comprising two light chains A, and (iii) a dimeric protein comprising two heavy chain Fc regions, (e) contacting the solution with a Protein L matrix at a certain conductivity so that the antibodies comprising at least one light chain A are bound to the Protein L matrix, and (f) eluting the bound antibodies from the Protein L matrix by lowering the conductivity. In further embodiments, the antibody described in (i) is a one-armed antibody, and the antibody described in (ii) is a whole antibody.

In the present invention, the proteins described in (i), (ii), and (iii) can work as a protein comprising at least one Protein L binding motif, a protein comprising at least two Protein L binding motifs, and a protein comprising no Protein L binding motifs, respectively. Since each of the proteins comprises a different number of the Protein L binding motifs, it can be expected that each of the proteins is separately eluted from the Protein L matrix, and as a result of that, the protein described in (i) is separated from the proteins described in (ii) and (iii).

In certain embodiments, the polypeptide A forms a multimeric (e.g. dimeric) protein, which comprises two or more of the polypeptides A. In particular embodiments, the invention provides a method comprising the steps of (a) culturing cells under conditions suitable for expression of a polypeptide A, (b) collecting a solution comprising at least two types of proteins expressed in the cells, which are (i) a protein comprising at least one polypeptide A, and (ii) a multimeric (e.g., dimeric) protein of the protein described in (i), (c) contacting the solution with a Protein L matrix at a certain conductivity so that the proteins are bound to the Protein L matrix, and (d) eluting the bound proteins from the Protein L matrix by lowering the conductivity.

In particular embodiments, the invention provides a method comprising the steps of (a) isolating a nucleic acid which encodes a polypeptide A, (b) transforming host cells with an expression vector comprising the nucleic acid, (c) culturing the host cells under conditions suitable for expression of the polypeptide A, (d) collecting a solution comprising at least two types of proteins expressed in the host cells, which are (i) a protein comprising at least one polypeptide A, and (ii) a multimeric (e.g., dimeric) protein of the protein described in (i), (e) contacting the solution with a Protein L matrix at a certain conductivity so that the proteins are bound to the Protein L matrix, and (f) eluting the bound proteins from the Protein L matrix by lowering the conductivity.

In some embodiments, a protein comprising at least one Protein L binding motif in the present invention is an antibody fragment. In certain embodiments, the antibody fragment is formed from a polypeptide comprising at least one Protein L binding motif (referred to as a polypeptide A). In certain embodiments, the antibody fragment associates into a multimeric (e.g. dimeric) protein, which comprises two or more of the antibody fragments. In further embodiments, the polypeptide A may additionally comprises at least one Protein L non-binding motif. In particular embodiments, the invention provides a method comprising the steps of (a) culturing cells under conditions suitable for expression of a polypeptide A, (b) collecting a solution comprising at least two types of proteins expressed in the cells, which are (i) an antibody fragment comprising at least one polypeptide A, and (ii) a multimeric (e.g., dimeric) protein of the antibody fragment described in (i), (c) contacting the solution with a Protein L matrix at a certain conductivity so that the proteins are bound to the Protein L matrix, and (d) eluting the bound proteins from the Protein L matrix by lowering the conductivity. In further embodiments, the antibody fragment is any one of scFv, diabody, scFv dimer, taFv, (scFv)2, scDb, scFab, TandAb, triabody, and tetrabody.

In particular embodiments, the invention provides a method comprising the steps of (a) isolating a nucleic acid which encodes a polypeptide A, (b) transforming host cells with an expression vector comprising the nucleic acid, (c) culturing the host cells under conditions suitable for expression of the polypeptide A, (d) collecting a solution comprising two types of proteins expressed in the host cells, which are (i) an antibody fragment comprising at least one polypeptide A, and (ii) a multimeric (e.g., dimeric) protein of the antibody fragment described in (i), (e) contacting the solution with a Protein L matrix at a certain conductivity so that the proteins are bound to the Protein L matrix, and (f) eluting the bound proteins from the Protein L matrix by lowering the conductivity. In further embodiments, the antibody fragment is any one of scFv, diabody, scFv dimer, taFv, (scFv)2, scDb, scFab, TandAb, triabody, and tetrabody.

In the present invention, the proteins described in (i) and (ii) can work as a protein comprising at least one Protein L binding motif, and a protein comprising at least two Protein L binding motifs, respectively. Since each of the proteins comprises a different number of the Protein L binding motifs, it can be expected that each of the proteins is separately eluted from the Protein L matrix, and as a result of that, the protein described in (i) is separated from the protein described in (ii).

Proteins comprising at least one Protein L binding motif produced by any of the above-mentioned methods are also included in the present invention.

In certain embodiments, Protein L used herein is immobilized onto a solid support or matrix for affinity purification of proteins of interest. A commercially available matrix with Protein L ligands is, for example, HiTrap™ Protein L (GE Healthcare), Capto™ L (GE Healthcare), Pierce™ Protein L Agarose (Thermo Scientific), Protein L-Agarose HC (ProteNova), TOYOPEARL(registered trademark) AF rProtein L-650F (Tosoh Bioscience), KanCap™ L (Kaneka), Protein L Resin (Genscript), MabAffinity(registered trademark) Protein L High Flow Beads (ACRO Biosystems), Amintra Protein L Resin (Expedeon), and ProL™ rProtein L Agarose Resin (Amicogen). Protein L variants such as an alkali-stabilized Protein L described in WO2016/096643 and WO2016/096644 or an affinity-increased Protein L can also be used as affinity ligands, so long as they maintain the immunoglobulin-binding ability Protein L originally has. Substances such as agarose, cellulose, dextran, polystyrene, polyacrylamide, polymethacrylate, latex, controlled pore glass, and spherical silica can be utilized as a matrix. Methods of binding affinity ligands to a matrix are well known in the purification art. See, e.g., Affinity Separations: A Practical Approach (Practical Approach Series), Paul Matejtschuk (Ed.), (Irl Pr: 1997); and Affinity Chromatography, Herbert Schott (Marcel Dekker, New York: 1997).

In the present invention, a protein bound to a Protein L matrix at a certain conductivity can be eluted from the Protein L matrix by lowering the conductivity. When two or more different proteins are present in a solution, each of which comprises a different number of the Protein L binding motifs, the proteins are expected to be eluted from the Protein L matrix in the ascending order of the number of the Protein L binding motifs, due to the difference of their binding affinity to Protein L. The conductivity value at which each of the proteins is eluted may not always be constant, but varying depending on other factors such as pH. In certain embodiments, a protein comprising at least one Protein L binding motif can be eluted from the Protein L matrix at a conductivity between 0.01 and 16 mS/cm. In certain embodiments, a protein comprising at least one Protein L binding motif can be eluted from the Protein L matrix during an elution step of lowering a conductivity from 16 to 0.01 mS/cm. The actual conductivity value at which a protein comprising at least one Protein L binding motif is eluted from the Protein L matrix can be, for example, a conductivity between 0.01 and 1 mS/cm, between 1 and 2 mS/cm, between 2 and 3 mS/cm, between 3 and 4 mS/cm, between 4 and 5 mS/cm, between 5 and 6 mS/cm, between 6 and 7 mS/cm, between 7 and 8 mS/cm, between 8 and 9 mS/cm, between 9 and 10 mS/cm, between 10 and 11 mS/cm, between 11 and 12 mS/cm, between 12 and 13 mS/cm, between 13 and 14 mS/cm, between 14 and 15 mS/cm, or between 15 and 16 mS/cm. In one illustrative embodiment, a protein comprising one Protein L binding motif can be eluted from the Protein L matrix at a conductivity, for example, between 2 and 16 mS/cm. The actual conductivity value at which a protein comprising one Protein L binding motif is eluted from the Protein L matrix can be, for example, a conductivity between 2 and 3 mS/cm, between 3 and 4 mS/cm, between 4 and 5 mS/cm, between 5 and 6 mS/cm, between 6 and 7 mS/cm, between 7 and 8 mS/cm, between 8 and 9 mS/cm, between 9 and 10 mS/cm, between 10 and 11 mS/cm, between 11 and 12 mS/cm, between 12 and 13 mS/cm, between 13 and 14 mS/cm, between 14 and 15 mS/cm, or between 15 and 16 mS/cm. In further embodiments, a protein comprising two Protein L binding motifs can be eluted from the Protein L matrix at a lower conductivity than a protein comprising one Protein L binding motif. In another illustrative embodiment, a protein comprising two Protein L binding motifs can be eluted from the Protein L matrix at a conductivity, for example, between 0.01 and 8 mS/cm. The actual conductivity value at which a protein comprising two Protein L binding motifs is eluted from the Protein L matrix can be, for example, a conductivity between 0.01 and 1 mS/cm, 1 and 2 mS/cm, 2 and 3 mS/cm, 3 and 4 mS/cm, 4 and 5 mS/cm, 5 and 6 mS/cm, 6 and 7 mS/cm, 7 and 8 mS/cm. A protein comprising no Protein L binding motifs is not expected to bind to the Protein L matrix and is expected to be eluted in the flow-through fraction or in the first washing step. In the present invention, the conductivity can be reduced in a gradient manner, in a stepwise manner, or in a combination of a gradient manner and a stepwise manner. The optimization of the elution condition is within the capability of a skilled person in the art.

In the conventional method of purifying a protein using Protein L, a protein is usually bound to a Protein L matrix at a certain pH and then eluted by lowering the pH. Meanwhile, in the present invention, a protein can be eluted from the Protein L matrix without changing the pH. In certain embodiments, the pH remains constant or substantially unchanged during the elution step of a protein comprising at least one Protein L binding motif from the Protein L matrix. In certain embodiments, a protein comprising at least one Protein L binding motif can be eluted from the Protein L matrix with the pH remaining constant or substantially unchanged between before and after the elution. In particular embodiments, a protein comprising at least one Protein L binding motif can be eluted from the Protein L matrix at an acidic pH. In particular embodiments, a protein comprising one Protein L binding motif and/or a protein comprising two Protein L binding motifs can be eluted from the Protein L matrix at an acidic pH. In further embodiments, the acidic pH is a pH below 7.0, for example, a pH higher than 1.0, 1.5, or 2.0 and lower than 3.5, 4.0, 4.5, 5.0, 5.5, 6.0, 6.5, or 7.0. In particular embodiments, the acidic pH is a pH between 2.4 and 3.3. In particular embodiments, the acidic pH is a pH such as, for example, 2.4, 2.5, 2.6, 2.7, 2.8, 2.9, 3.0, 3.1, 3.2, and 3.3.

The term “conductivity” refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is mmhos (mS/cm). The conductivity of a solution may be altered by changing the concentration of ions therein. For example, the concentration of a buffering agent and/or a salt (e.g., NaCl or KCl) in the solution may be altered in order to achieve the desired conductivity. The actual value of conductivity can be measured using a commercially-available conductivity meter sold, e.g., by HORIBA.

In the present invention, separation of a desired protein comprising a certain number of the Protein L binding motifs from other proteins (byproducts) comprising different numbers of the Protein L binding motifs can be expected as one of the possible effects. The concentration of the desired protein can also be expected to increase relative to the concentration of the byproducts in a composition as compared to before the purification. In some embodiments, the purity and/or proportion of the protein after the elution from the Protein L matrix is, for example, 70% or more, 75% or more, 80% or more, 85% or more, 90% or more, 95% or more, or 98% or more. The purity and/or proportion of the protein can be determined by a variety of art-recognized analytical methods such as hydrophobic interaction-high performance liquid chromatography (HIC-HPLC), ion exchange-high performance liquid chromatography (IEX-HPLC), cation exchange-high performance liquid chromatography (CEX-HPLC), reverse phase-high performance liquid chromatography (RP-HPLC), SDS-PAGE, immunoblotting, capillary electrophoresis (CE)-SDS, or isoelectric focusing (IEF). Alternatively, it can also be determined by a method described in Example 4 of the present invention.

### EXAMPLES

The following are examples of methods and compositions of the invention. It is understood that various other embodiments may be practiced, given the general description provided above.

### Example 1: Generation of Recombinant Antibodies

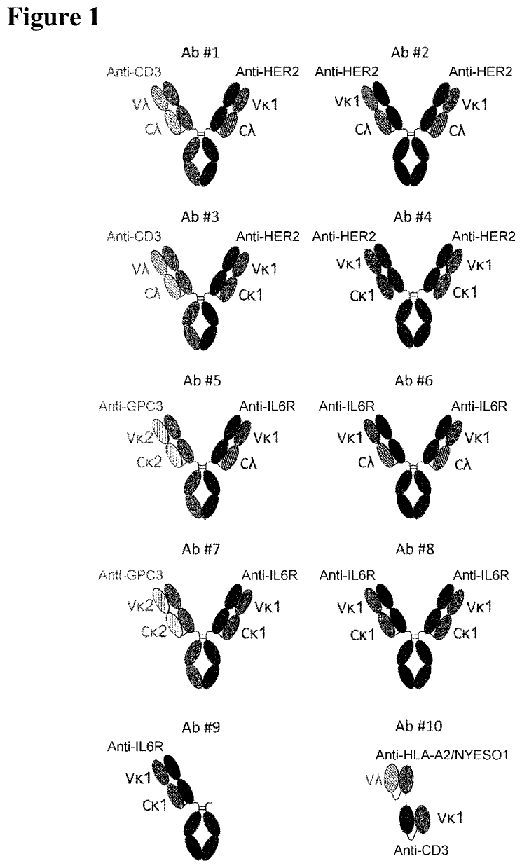

Recombinant antibodies described in Table 1 and FIG. 1 were generated using conventional methods published elsewhere. These includes transient expression with mammalian cells such as FreeStyle293-F cell line or Expi293 cell line (Thermo Fisher) and purification using protein A as well as gel filtration chromatography. The formulation of purified antibodies was 1× D−PBS(−), whose conductivity was around 15.4 mS/cm.

Functional and structural properties of the antibodies generated

| Antibody | Antibody | Arm-A | | Arm-B | |
|---|---|---|---|---|---|
| ID | type | antigen | LC type | antigen | LC type |
| #1 | Bispecific Ab | HER2 | Vκ1-Cλ | CD3* | Vλ-Cλ |
| #2 | Monospecific Ab | HER2 | Vκ1-Cλ | HERZ | Vκ1-Cλ |
| #3 | Bispecific Ab | HER2 | Vκ1-Cκ | CD3 | Vλ-Cλ |
| #4 | Monospecific Ab | HER2 | Vκ1-Cκ | HERZ | Vκ1-Cκ |
| #5 | Bispecific Ab | IL6R | Vκ1-Cλ | GPC3 | Vκ2-Cκ |
| #6 | Monospecific Ab | IL6R | Vκ1-Cλ | IL6R | Vκ1-Cλ |
| #7 | Bispecific Ab | IL6R | Vκ1-Cκ | GPC3 | Vκ2-Cκ |
| #8 | Monospecific Ab | IL6R | Vκ1-Cκ | IL6R | Vκ1-Cκ |
| #9 | One arm Ab | IL6R | Vκ1-Cκ | — | — |
| #10 | BiTE | HLA-A2/ | Vλ | CD3^{‡} | Vκ1 |
| | | NYESO1 | | | |

*Arm B of antibodies #1 and #3 are targeting N-tenninal peptide of human CD3ε.
^{‡}Arm B of antibody #10 is targeting conformational epitope of human CD3εγ or CD3εδ heterodimer.

### Example 2: Measurement of Conductivity and pH

Conductivity was measured using conductivity meter (HORIBA scientific, B-771 COND) or pH/ORP/COND METER (HORIBA scientific, D-74) while pH was measured using pH meter (Mettler Toledo, S220-Bio) or pH/ION METER (HORIBA scientific, F-72). Conductivity and pH were also monitored with the Unicorn software (GE Healthcare) that accompanies with the purification system, such as AKTA Avant25 or AKTAexplorer 10S (GE Healthcare).

### Example 3: Systems for Protein L Purification

AKTA Avant25 or AKTAexplorer 10S (GE Healthcare) connected with 0.7×2.5 cm Protein L-Agarose HC (ProteNova, P-044-1-5) column [column volume (CV)=1 mL] or 0.7×2.5 cm HiTrap Protein L (GE Healthcare, 29-0486-65) column [column volume (CV)=1 mL], were used in Examples 5 to 12 or in Example 13, respectively, to conduct Protein L purifications at the flow rate of 1 mL/min. Buffers and acids used are described in each section.

### Example 4: Protein Analysis Method for Identification of Monospecific and Bispecific Antibodies

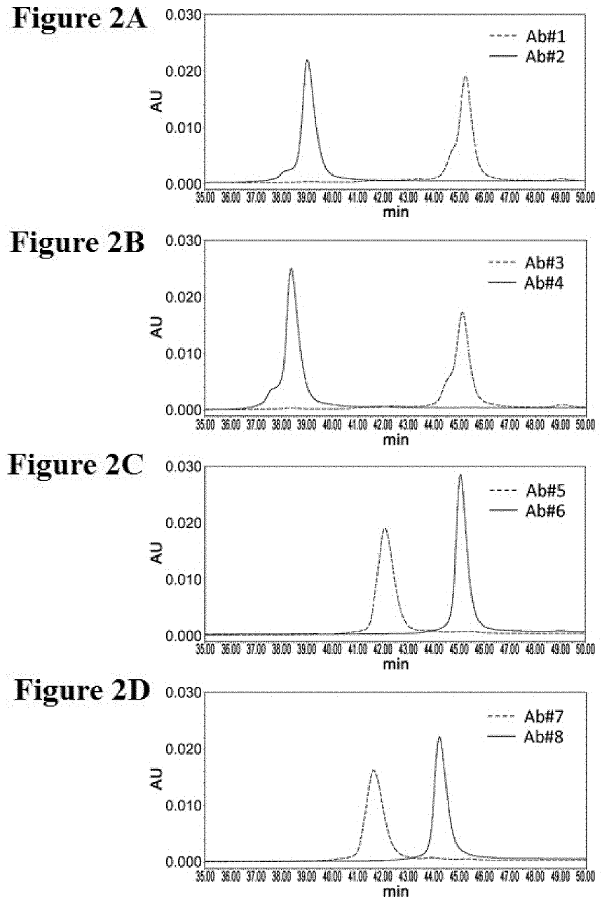

Cation exchange chromatography (CIEX) was carried out on a ProPac™ WCX-10 LC Column, 10 micro m, 4 mm×250 mm (Thermo Fisher) at a flow rate of 0.5 ml/min on an Alliance HPLC system (Waters). Column temperature was set at 40 degrees C. 4 micro g of samples were loaded after column was equilibrated with mobile phase A (CX-1 pH Gradient Buffer A, pH5.6, Thermo Fisher). Then the column was eluted with linear gradient from 0 to 100% mobile phase B (CX-1 pH Gradient Buffer B, pH 10.2, Thermo Fisher) for 50 minutes. Detection was done by UV detector (280 nm). As shown in FIG. 2, the retention times of the peaks representing antibodies #1 and #2, #3 and #4, #5 and #6, and #7 and #8 were clearly distinct from each other, respectively. This allowed to identify each antibody among the respective pairs. Percentage of each antibody in the Protein L eluates was calculated from the area of each peak.

### Example 5: Separation of Antibodies with One and Two V Kappa 1 by Lowering Conductivity Under Various Acidic pH (Part 1)

Antibody #1 is a bispecific antibody that has anti-HER2 with chimeric V kappa 1-C lambda light chain (LC) on one arm while another arm has anti-CD3 with lambda LC. In addition, antibody #2 is a monospecific antibody having anti-HER2 with chimeric V kappa 1-C lambda LC on both arms. Since there are only a few residues in the constant region of kappa LC that has contact with Protein L, it is reasonable to think that the variable region of kappa LC is enough for Protein L binding [1]. Therefore, antibody #1 has one binding site toward Protein L while antibody #2 has two.

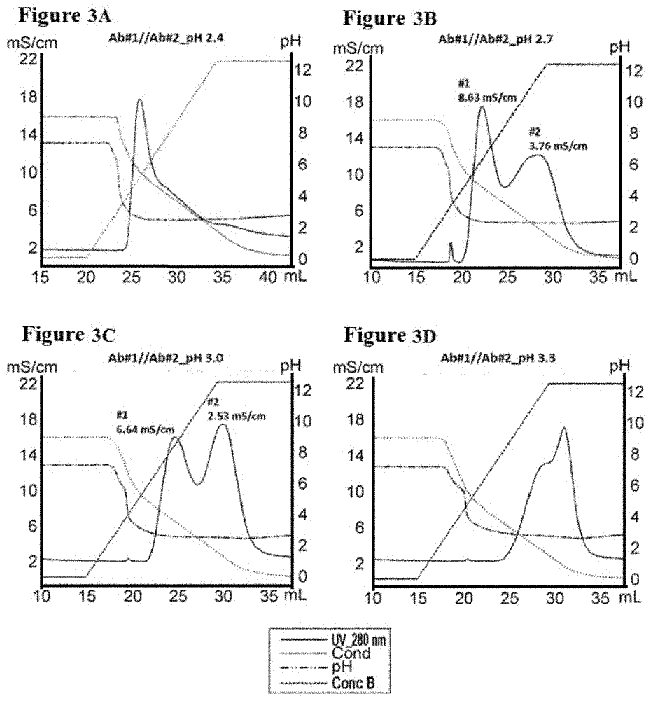

In order to test if we can separate antibody #1 and #2 by using the difference of monovalent versus bivalent binding, respectively, to Protein L-conjugated resin, 0.5 mg each of antibodies #1 and #2 were mixed and applied to a Protein L column. After washing the column with 1×D-PBS(−), 15 CV of linear gradient elution was conducted under one of the four conditions as follows: [pH 2.4+/−0.1] 500 mM Na-acetate, 100 mM NaCl, 11.34+/−0.01 mS/cm (buffer A1) to 500 mM Na-acetate, 1.12+/−0.01 mS/cm (buffer B1); [pH 2.7+/−0.1] 150 mM Na-acetate, 100 mM NaCl, 11.22+/−0.05 mS/cm (buffer A2) to 150 mM Na-acetate, 0.65+/−0.01 mS/cm (buffer B2); [pH 3.0+/−0.1] 50 mM Na-acetate, 100 mM NaCl, 11.02+/−0.07 mS/cm (buffer A3) to 50 mM Na-acetate, 0.37+/−0.01 mS/cm (buffer B3); [pH 3.3+/−0.1] 20 mM Na-acetate, 100 mM NaCl, 11.12+/−0.07 mS/cm (buffer A4) to 20 mM Acetic acid, 0.22+/−0.00 mS/cm (buffer B4). As a result, two distinct protein peaks were observed when tested under pH 2.7 and pH 3.0 (FIGS. 3B and 3C). By analyzing the protein identity of each of the peak fractions with CIEX, for both pH conditions, the first peak represented antibody #1 that binds to Protein L in a monovalent manner while the second peak represented antibody #2 that binds to Protein L in a bivalent manner. On the other hand, the separation was not as clear at pH 2.4 and pH 3.3 (FIGS. 3A and 3D). Thus, this result suggested that antibodies binding to Protein L column in monovalent and bivalent manner can be eluted separately by decreasing the conductivity from around 11.34 mS/cm to 0.22 mS/cm within a range of acidic pH, i.e. between pH 2.4 and pH 3.3.

### Example 6: Stepwise Separation of Antibodies with One and Two Vkappa 1 by Different Conductivity Under Acidic pH (Part 1)

Since the separation of bispecific (#1) and monospecific (#2) antibodies was observed by gradually lowering conductivity under pH 2.7 and pH 3.0 (FIG. 3), to simplify the method for more practical use, separation by stepwise elution was further evaluated. In order to perform stepwise elution at pH 2.7 and pH 3.0, the conductivity for the first elution step was set at around the measured conductivity from the first peak during the linear gradient elution under respective pH as shown in FIG. 3. This first elution step was meant to elute antibodies with a monovalent binding to Protein L. The elution buffers used for the first step were 70% buffer A2 mixed with 30% buffer B2 (around 8.50 mS/cm, pH 2.7) or 55% buffer A3 mixed with 45% buffer B3 (around 6.66 mS/cm, pH 3.0) respectively. The elution buffer for the second step was buffer B2 (pH 2.7) or buffer B3 (pH 3.0), respectively, which aim to elute antibodies binding to Protein L in a bivalent manner.

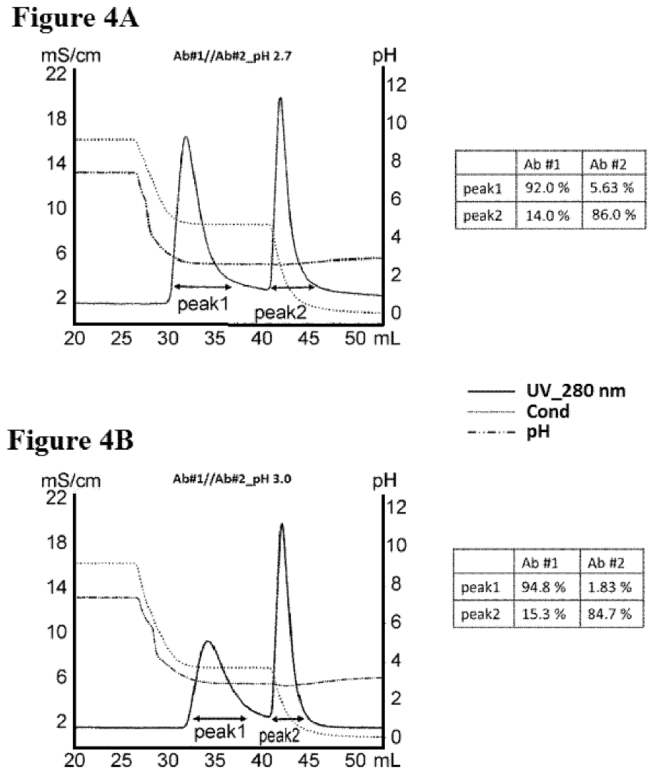

A mixture of antibodies #1 and #2 (0.5 mg each) was loaded onto a Protein L column, washed with 1×D-PBS(−), then the stepwise elution was conducted for 15 CV per step using elution buffers as described above. Peaks that appeared at each step under pH 2.7 and pH 3.0 were analyzed with the CIEX method. As a result, the peaks from the first elution step contained from 92.0 to 94.8% of antibody #1 with trace amount of antibody #2 (from 1.8 to 5.6%) while the peaks from the second elution step contained mainly antibody #2 (from 84.7 to 86.0%) with some antibody #1 (from 14.0 to 15.3%) (FIG. 4). Therefore, this result clearly demonstrates that antibodies binding to Protein L column in monovalent and bivalent manner can be eluted separately by stepwise elution using different conductivity at low pH.

### Example 7: Separation of Antibodies with One and Two Full Length Kappa LC by Lowering Conductivity Under Acidic pH (Part 1)

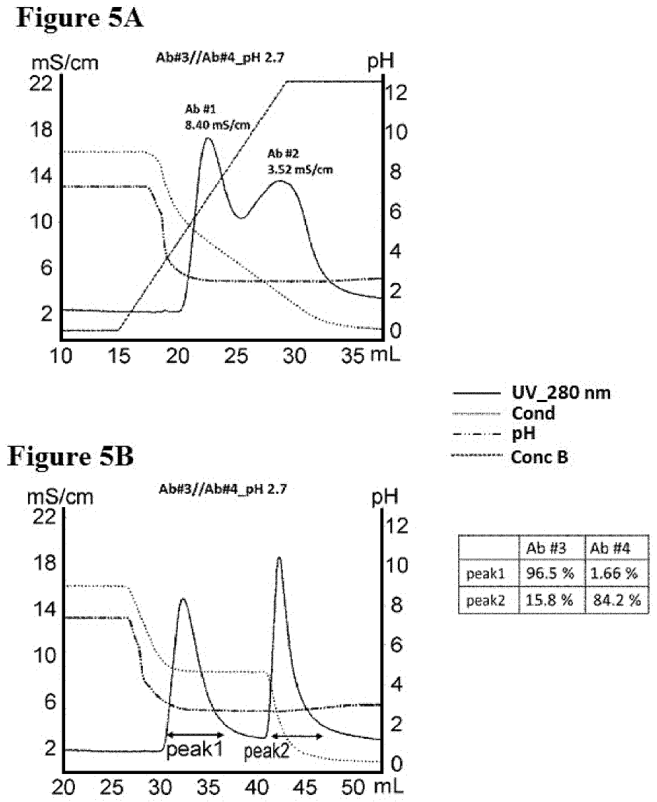

In order to confirm that full length kappa 1 LC behaves the same as V kappa 1-C lambda chimeric LC, anti-CD3/anti-HER2 bispecific antibody (antibody #3) and anti-HER2 monospecific antibody (antibody #4) with full length kappa 1 LC instead of V kappa 1-C lambda chimeric LC as in antibodies #1 and #2, respectively, were prepared and evaluated with Protein L separation. Since it was assumed that the result would be similar to the results obtained using antibodies #1 and #2 (FIG. 3), only pH 2.7 was tested with the gradient elution under the condition described in Example 5. As expected, two peaks were observed and the CIEX analysis showed that the first peak represented antibody #3 and the second peak antibody #4 (FIG. 5A).

Next, stepwise elution was tested under pH 2.7 as done in Example 6. The elution buffer for the first step was 70% buffer A2 mixed with 30% buffer B2 (around 8.50 mS/cm, pH 2.7) and the elution buffer for the second step was 100% buffer B2. As a result, a peak was observed at each step: peak 1 contained 96.5% of antibody #3 while peak 2 contained mainly antibody #4 with percentage of 84.2 (FIG. 5B). This result was comparable to Example 6 and was a reasonable result as both antibody #3 with full length kappa 1 LC and antibody #1 with chimeric V kappa 1-C lambda LC are the same in terms of the number of binding surface to Protein L. Therefore the separation of monovalent and bivalent binding to Protein L can be performed similarly between full length kappa 1 LC and chimeric V kappa 1-C lambda LC.

### Example 8: Separation of Antibodies with One and Two V Kappa 1 by Lowering Conductivity Under Various Acidic pH (Part 2)

In order to confirm the result obtained from Examples 5 and 6, another set of antibodies were prepared: a bispecific antibody that has chimeric V kappa 1-C lambda LC (anti-IL6R) on one arm while another arm has kappa 2 LC (anti-GPC3) (antibody #5), and a monospecific antibody having anti-GPC3 with chimeric V kappa 1-C lambda LC on both arms (antibody #6). Of note, kappa 2 LC is known not to be able to bind to Protein L [1], therefore antibody #5 can bind to Protein L in a monovalent fashion while antibody #6 can bind in a bivalent fashion.

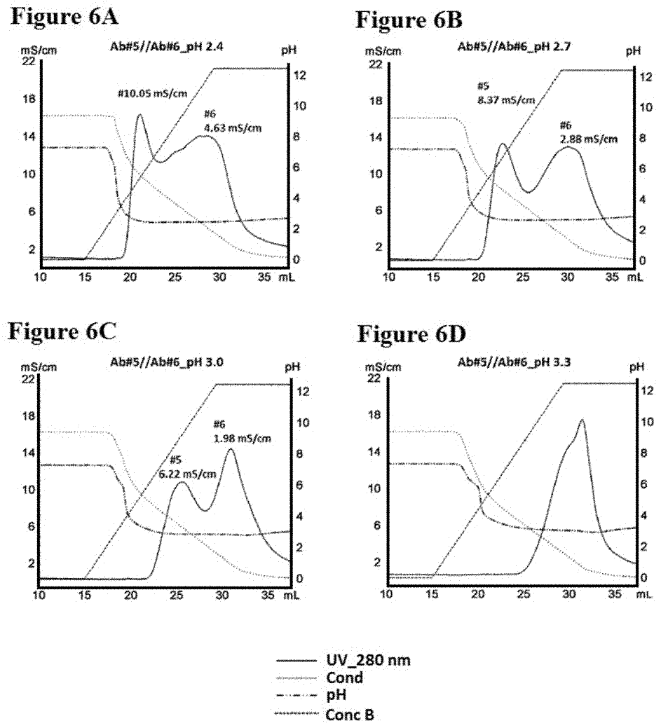

Similar to Example 5, 0.5 mg each of antibody #5 and #6 were mixed and applied to a Protein L column, washed with 1×D-PBS(−), and eluted with 15 CV of linear conductivity gradient elution under four different pH as described. As a result, two distinct protein peaks were observed when tested under pH 2.4, 2.7 and pH 3.0, while pH 3.3 showed less separation (FIG. 6). By analyzing the protein identity of each of the peak fractions by CIEX, for three pH that showed clear separation, the first peak represented antibody #5 that binds to Protein L in a monovalent manner while the second peak represented antibody #6 that binds to Protein L in a bivalent manner. Thus, this result support the result obtained in Example 5 that antibodies binding to Protein L column in monovalent and bivalent manner can be eluted separately by decreasing the conductivity from around 11.34 mS/cm to 0.22 mS/cm within a range of acidic pH, i.e. between pH 2.4 and pH 3.3.

### Example 9: Stepwise Separation of Antibodies with One and Two V Kappa 1 by Different Conductivity Under Acidic pH (Part 2)

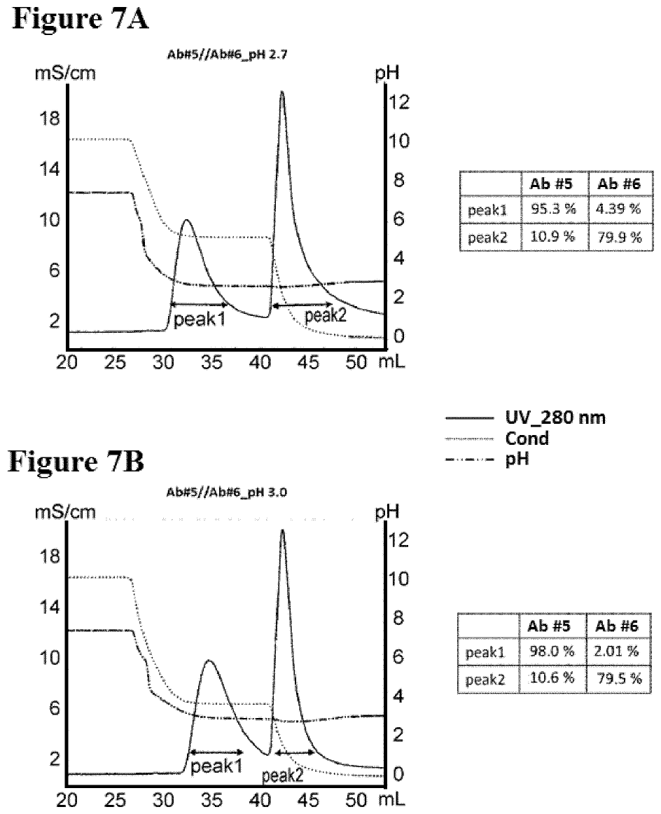

In order to confirm that stepwise separation is also possible with the antibody pair of #5 and #6, stepwise elution at pH 2.7 and pH 3.0 was further evaluated. Following the method described in Example 6 with consideration of the conductivity value of the first peak in the conductivity gradient experiments (FIG. 6), 70% buffer A2/30% buffer B2, pH 2.7 (around 8.54 mS/cm) or 50% buffer A3/50% buffer B3, pH 3.0 (around 6.13 mS/cm), respectively, was used for the first elution step while buffer B2 or buffer B3, respectively, was used for the second elution step. As a result, under both pH, the peaks from the first elution step contained antibody #5 in high purity (from 95.3 to 98.0%), while the peaks from the second elution step contained mainly antibody #6 (around 80%) (FIG. 7). Therefore, this result further confirmed that antibodies binding to Protein L in a monovalent manner can be separated from that in a bivalent manner by stepwise elution with different conductivity at low pH.

### Example 10: Separation of Antibodies with One and Two Full Length Kappa LC by Lowering Conductivity Under Acidic pH (Part 2)

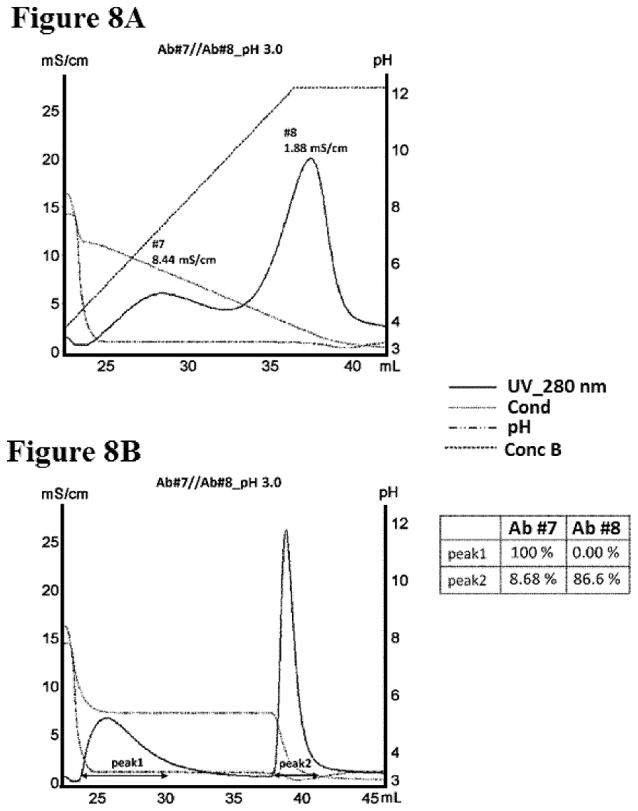

A same set of experiment as Example 7 was conducted by using anti-IL6R/anti-GPC3 bispecific antibody (antibody #7) and anti-IL6R monospecific antibody (antibody #8) with full length kappa 1 LC instead of V kappa 1-C lambda chimeric LC as in antibody #5 and #6, respectively, at pH 3.0. As expected, two peaks were observed and the CIEX analysis showed that the first peak represented antibody #7 and the second peak antibody #8 (FIG. 8A).

Next, stepwise elution was tested under the same pH. The elution buffer for the first step was 50 mM Na-acetate, 60 mM NaCl, pH 3.0 (7.40 mS/cm) while the elution buffer for the second step was buffer B3. As a result, as expected, a peak was observed at each step: peak 1 contained around 100% of antibody #7 while peak 2 contained mainly antibody #8 with the percentage of 86.6 (FIG. 8B). This supports the concept again that separation of monovalent and bivalent binding to Protein L can be performed similarly between full length kappa 1 LC and chimeric V kappa 1-C lambda LC.

### Example 11: Separation of One-Arm and Two-Arm Antibodies with Full Length Kappa LC by Lowering Conductivity Under Acidic pH

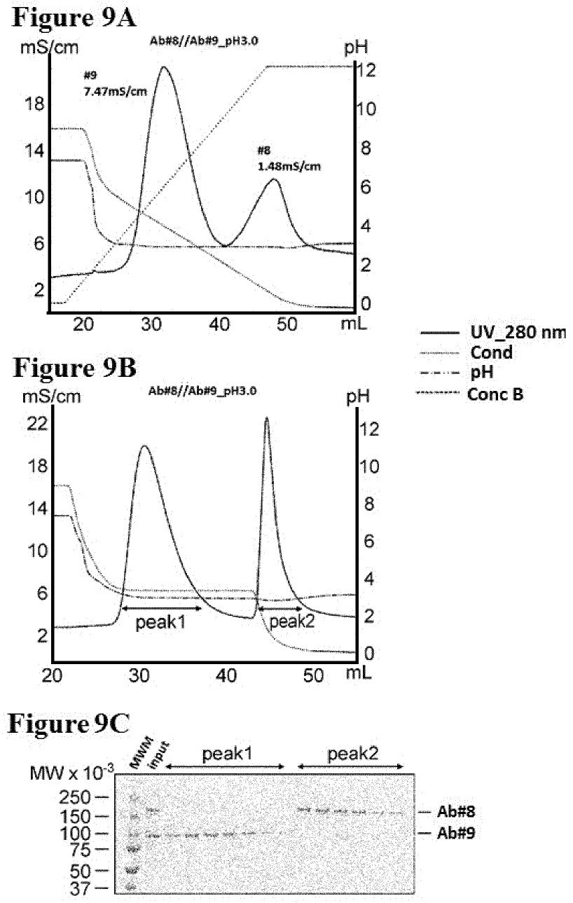

In the present invention, difference in number of Protein L binding sites are critical for separation. In such situation, one-arm antibody having one Protein L binding site and two-arm antibodies having two Protein L binding sites should also be able to separate under the same concept. In order to test this, 0.5 mg each of one-arm anti-IL6R antibody (antibody #9) and two-arm anti-IL6R monospecific antibody (antibody #8) with full length kappa 1 LC were mixed and applied to a Protein L column, washed with 1×D-PBS(−), and eluted with 30 CV of linear conductivity gradient elution at pH 3.0 under the same buffer condition used in Example 5. As a result, as expected, two peaks were observed: the first peak appeared at 7.47 mS/cm while the second peak appeared at 1.48 mS/cm (FIG. 9A). Since the molecular weight of one-arm and two-arm antibodies are different (˜1×10E5 vs 1.5×10E5, respectively), fractions from each peak were analyzed with SDS-PAGE under non-reducing condition. The SDS-PAGE analysis showed that the first peak represented antibody #9 while the second peak represented antibody #8 (data not shown).

Next, stepwise elution was tested under the same pH. The elution buffer used for the first step was 50 mM Na-acetate, 50 mM NaCl, pH 3.0 (around 6.23 mS/cm) while the elution buffer for the second step was buffer B3. As a result, a peak was observed at each step (FIG. 9B): according to the non-reducing SDS-PAGE analysis of fractions from peaks 1 and 2, as expected, peak 1 exclusively contained one-arm antibody #9 while peak 2 mainly contained two-arm antibody #8 (FIG. 9C). This supports the concept again that separation of monovalent and bivalent binding to Protein L can be performed similarly between one-arm and two-arm antibodies.

### Example 12: Separation of Monomeric and Oligomeric BiTE Antibodies by Lowering Conductivity Under Acidic pH

BiTE antibodies are fusion proteins consisting of two single-chain variable fragments (scFv) of different antibodies. With having V kappa 1 domain in scFv, BiTE can also be able to purify by Protein L. On the other hand, since BiTE antibodies do not have Fc domain, it is usually not purified by Protein A affinity chromatography. It is known that upon expression BiTE antibodies tend to form aggregate at certain rate, therefore a convenient method to separate monomers and aggregates is desired. In order to test if Protein L column can separate monomeric and oligomeric BiTE antibodies by gradually lowering the conductivity at low pH, the BiTE antibody which has one scFv with V kappa 1 domain and the other scFv with V lambda domain (antibody #10) was prepared. It should be noted that the BiTE antibody used in the present invention in monomeric form has single Protein L binding site while oligomeric BiTE antibody contains more than two protein L binding sites in one aggregate.

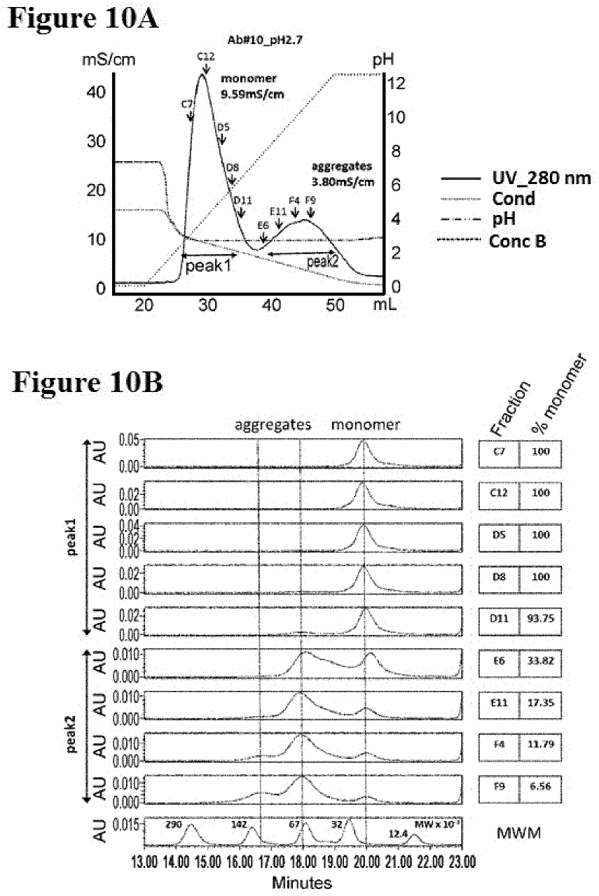

In order to test this, 1.0 mg of purified BiTE antibodies containing 21.93% oligomer was applied to a Protein L column. After washing the column with 1×D-PBS(−), 30 CV of linear gradient elution was conducted under pH 2.7 using following buffers: 150 mM Na-acetate, 100 mM NaCl, 11.22+/−0.05 mS/cm (buffer A2) and 150 mM Na-acetate, 0.65+/−0.01 mS/cm (buffer B2). As a result, two distinct protein peaks were observed (FIG. 10A). Since there are clear size differences between monomer and oligomers, fractions from peaks 1 and 2 were analyzed by SEC-HPLC using TSKgel G3000SW_{XL} column (Tosoh Bioscience). By calculating the peak area, fractions from peak 1 contained monomeric BiTE antibody with high purity (93.75 to 100%), while peak 2 consisted mainly of oligomers with some monomers (FIG. 10B). Thus, this result clearly demonstrated that Protein L separation with lowering the conductivity at low pH can separate monomeric and oligomeric forms of BiTE antibodies. This result suggests that the method described in the present invention can also be applied for separation of monomer and oligomer of any other protein that contains one Protein L binding site per molecule.

### Example 13: Use of Different Protein L Conjugated Column

In Examples 5 to 12, separation experiments were conducted using Protein L conjugated resin named Protein L-Agarose HC from ProteNova. In order to confirm that the Protein L separation method in the present invention can be applied similarly to different Protein L conjugated columns universally, the separation of antibodies #5 and #6 were conducted with different Protein L column: GE Healthcare's HiTrap Protein L. Of note, the amino acid sequence of Protein L used in ProteNova's and GE Healthcare's resin may be slightly different as at least the sequence of ProteNova's Protein L should be engineered for adding alkali resistance.

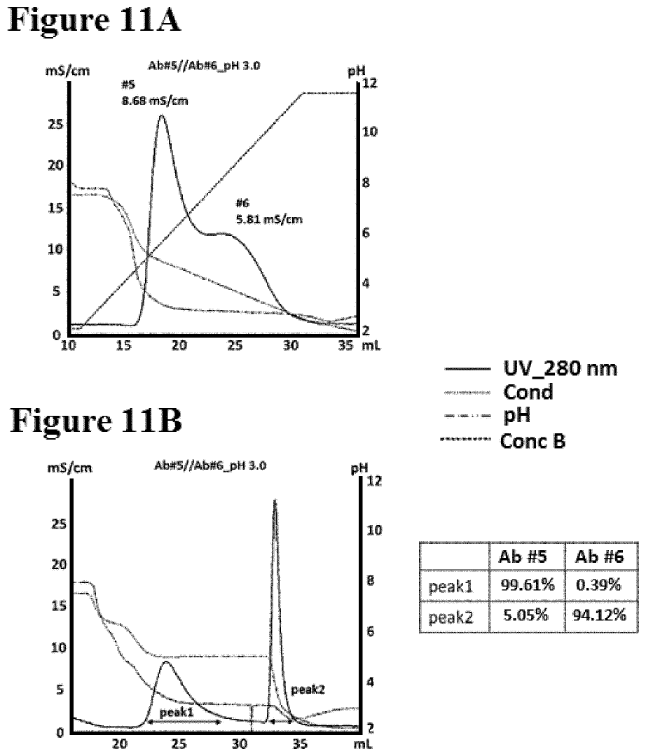

In order to test if HiTrap Protein L can be used for separation, 0.5 mg each of antibodies #5 and #6 were mixed and applied to the HiTrap Protein L column, washed with 1×D-PBS(−), and eluted with 20 CV of linear conductivity gradient elution under pH 3.0 using the same buffer in Example 5. As a result, two distinct protein peaks were observed (FIG. 11A). By analyzing the protein identity of each of the peak fractions by CIEX, the first peak at 8.68 mS/cm represented antibody #5 that binds to Protein L in a monovalent manner while the second peak at 5.81 mS/cm represented antibody #6 that binds to Protein L in a bivalent manner. As a next step, with consideration of the conductivity value of the first peak in the conductivity gradient experiments (FIG. 11A), 75% buffer A3/25% buffer B3, pH 3.0 (around 8.86 mS/cm) was used for the first elution step while buffer B3 was used for the second elution step. As a result, the peaks from the first elution step contained antibody #5 in high purity (from 99.61%), while the peaks from the second elution step contained mainly antibody #6 (around 94%) (FIG. 11B). Therefore, this result suggested that the Protein L separation using different conductivity at low pH can be performed by variety of Protein L conjugated resins.

### REFERENCE

1. Graille, M., Stura, E. A., Housden, N. G., Beckingham, J. A., Bottomley, S. P., Beale, D., Taussig, M. J., Sutton, B. J., Gore, M. G., and Charbonnier, J. (2001) Structure 9, 679-687

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of purifying a protein comprising the step of eluting at least two different proteins from a Protein L matrix by lowering a conductivity, wherein each of the proteins comprises a different number of Protein L binding motifs.

2. The method of claim 1, wherein one of the proteins which comprises a certain number of Protein L binding motifs is separated from the other protein(s) in the elution step.

3. The method of claim 1, wherein the Protein L binding motif is an antibody kappa chain variable region or a fragment thereof which has a binding ability to Protein L.

4. The method of claim 3, wherein the antibody kappa chain variable region is selected from the group consisting of human variable kappa subgroup 1 (VK1), human variable kappa subgroup 3 (VK3), human variable kappa subgroup 4 (VK4), mouse variable kappa subgroup 1 (VK1), and variants thereof.

5. The method of claim 1, wherein any one of the proteins is an antibody.

6. The method of claim 5, wherein the antibody is a whole antibody or an antibody fragment.

7. The method of claim 5, wherein the antibody is a monospecific antibody or a multispecific antibody.

8. The method of claim 5, wherein the at least two different proteins comprise:
(i) an antibody comprising two light chains, one of which comprises a Protein L binding motif, and the other of which comprises a Protein L non-binding motif, and
(ii) an antibody comprising two light chains, both of which comprise a Protein L binding motif.

9. The method of claim 1, wherein at least one of the proteins is eluted from the Protein L matrix at a conductivity between 0.01 and 16 mS/cm.

10. The method of claim 1, wherein the conductivity is reduced in a gradient manner or in a stepwise manner during the elution step.

11. The method of claim 1, wherein at least one of the proteins is eluted from the Protein L matrix at an acidic pH.

12. The method of claim 11, wherein at least one of the proteins is eluted from the Protein L matrix at a pH between 2.4 and 3.3.

13. The method of claim 11, wherein the pH remains constant or substantially unchanged during the elution step.

14. A method of producing a protein comprising the steps of:
(a) eluting at least two different proteins from a Protein L matrix by lowering a conductivity, and
(b) collecting one of the eluted proteins,
wherein each of the proteins comprises a different number of Protein L binding motifs.

15. An antibody comprising a light chain, which comprises a kappa variable region and a lambda constant region.
